(19) United States
(12) Patent Application Publication
(10) Pub. No.: US 2012/0190707 A1
(43) Pub. Date: Jul. 26, 2012

(54) SELECTIVE INHIBITORS OF AKT AND METHODS OF USING SAME

(76) Inventors: Ze'ev Ronai, La Jolla, CA (US); Maurizio Pellecchia, San Diego, CA (US); Gary Chiang, La Jolla, CA (US)

(21) Appl. No.: 13/357,541

(22) Filed: Jan. 24, 2012

Related U.S. Application Data
(63) Continuation-in-part of application No. 12/645,313, filed on Dec. 22, 2009.
Application No. 12/645,313 is a continuation-in-part of application No. 11/817,764, filed on Apr. 25, 2008.
(60) Provisional application No. 61/139,753, filed on Dec. 22, 2008.

(51) Int. Cl. A61K 31/4709; C07D 471/04; C07D 401/10; A61K 31/4704; A61P 35/00; A61P 25/28; C07D 401/06; C07D 215/227

(52) U.S. Cl. 514/300; 546/156; 546/113; 514/312

(57) ABSTRACT

The present invention describes an improved method for screening compounds for activity in inhibiting the enzymatic activity of Akt1 protein kinase. In general, the method comprises: (1) providing a plurality of compounds suspected of having Akt1 kinase inhibitory activity; (2) modeling the docking of each of the plurality of the compounds with a target binding site; (3) ranking the docked compounds by goodness of fit; (4) further selecting compounds; (5) optionally, visually analyzing structures of compounds selected in step (4) to remove any compounds with improbable docking geometry; and (6) experimentally testing the selected compounds from step (4) or step (5), if step (5) is performed, to determine their inhibitory activity against Akt1. The invention also encompasses pharmaceutical compositions including compounds whose inhibitory activity against Akt1 is discovered by the screening method, as well as methods of use of the pharmaceutical compositions to prevent and treat cancer and other conditions.

### RELATED APPLICATIONS

This application is a continuation-in-part of and claims priority to U.S. application Ser. No. 12/645,313, filed Dec. 22, 2009, which is a continuation-in-part of and claims priority to U.S. application Ser. No. 11/817,764, filed Apr. 25, 2008, and U.S. provisional application Ser. No. 61/139,753, filed Dec. 22, 2008. The disclosures of the above referenced applications are incorporated by reference in their entireties herein.

### FIELD OF THE INVENTION

This application is directed to screening methods for Protein Kinase B inhibitors, particularly screening methods employing virtual docking approaches, and compounds and compositions discovered by the use of these docking methods.

### BACKGROUND OF THE INVENTION

Protein phosphorylation plays a central role in many cellular events such as proliferation, differentiation, survival, and angiogenesis (Klingmuller, 1997). Consequently, unregulated kinase activity can result in uncontrolled cellular growth and inappropriate regulation of apoptosis, which is a key mechanism in oncogenesis suppression (Lev, et al., 2004).

Within this scenario AKT, also known as protein kinase B (PKB), has recently caught scientists' attention, since its aberrant activation has been recognized to be responsible for a wide range of proliferative and antiapoptotic processes in many human tumors (Dickson and Rhodes, 2004). The AKT signaling pathway is among the key tumor survival mechanisms. AKT is a subfamily consisting of three different cellular isoforms, namely, Akt1 (PKBR), Akt2 (PKBβ), and Akt3 (PKBγ). Akt1 is mostly involved in breast cancer and in gastric adenocarcinomas; Akt2 is amplified in ovarian, pancreatic, and breast cancers; and Akt3 is amplified in breast cancer and prostate cell lines (Okano, et al., 2000).

Akt1 is composed of a kinase domain, a N-terminal pleckstrin homology (PH) domain, and a short carboxyterminal tail region. This protein is activated when Thr308 and Ser473 are phosphorylated (Chijiwa, et al., 1990). Once activated, Akt1 inhibits apoptosis and stimulates cell cycle progression by phosphorylating numerous targets in various cell types, including cancer cells. Consequently, the development of molecules capable of blocking protein kinase B activity is a valuable route for anticancer drug discovery (Stratford, et al., 2004; Baxter, et al., 2000; Carr and Jhoti, 2002; Perola, et al., 2000).

AKT activation is mediated by phosphatidylinositol 3-OH kinase (PI3K) phosphorylates phosphatidylinositol-4,5-biphosphate (PIP2) to produce phosphatidylinositol-3,4,5-triphosphate (PIP3), which recruits AKT to the plasma membrane where Akt Ser-473 is phosphorylated by mammalian target of rapamycin (mTOR) or integrin linked kinase (ILK). Additional phosphorylation of Thr-308 at the catalytic site by the pyruvate dehydrogenase kinase isozyme 1 (PDK1) or the mTORC2 complex is needed for AKT activity. The PI3K activity in AKT activation is counterbalanced by phosphatase and tensin homologue deleted from chromosome 10 (PTEN). Therefore, inactivation of PTEN, a common occurrence in human cancer, results in constitutively high level of AKT activity (Carracedo and Pandolfi, 2008; Yuan and Cantley, 2008).

One of the proteins phosphorylated by activated Akt1 is the protein known as BAD, which normally encourages cells to undergo programmed cell death, or apoptosis. Once phosphorylated, BAD binds to a cytosolic protein designated 14-3-3, which inactivates BAD. Akt1 also promotes cell survival by inhibiting other cell death activators; one route for accomplishing this is by inhibition of transcription of the genes encoding the cell death activators, such as those of the Forkhead family, which are gene regulatory proteins that stimulate the transcription of genes that encode proteins that promote apoptosis.

Following its activation, AKT phosphorylates close to 100 substrates, through which it modulates a variety of cellular functions. Those include AKT's ability to elicit an antiapoptotic effect through the phosphorylation and inhibition of key pro-apoptotic proteins, such as BAD, MDM2 and members of the Forkhead family; the support of cell proliferation by inactivating p27 and inhibition of glycogen synthase kinase 3 (GSK3)-mediated Myc and cyclin D1 inhibition; the effect on growth, metabolism and angiogenesis; and lastly, on protein translation and ribosome biogenesis. AKT increases translational machinery to produce ribosomes and increases the protein synthesis rate by dual regulation of the GTPase-activating protein (GAP) TSC2 and PRAS40 (a proline-rich AKT substrate of 40 KDa).

AKT activity, often measured by its phosphorylation at Ser473, has been linked to poor prognosis in several different cancers, including melanoma, acute myelogenous leukemia, lung, head and neck, breast, endometrial, brain, gastric, ovarian, colon and prostate cancer (Cicenas, 2008; Dai et al., 2005). The tumor promoting activities elicited by AKT have raised the notion that AKT may serve as an important target for cancer treatment (Garcia-Echeverria and Sellers, 2008). Accordingly, growing efforts are devoted to developing inhibitors to AKT. Of those developed so far, many were designed against the Pleckstrin Homology (PH) domain of AKT or the ATP-binding domain (Carnero et al., 2008; Lindsley et al., 2008).

AKT activation in melanoma is reported to occur in about 50% of cases, where only a portion of these (20-30%) are attributed to PTEN mutations (Goel et al., 2006; Haluska et al., 2006; Robertson, 2005). Activated AKT cooperates with the B-Raf, which is mutated in 70% of melanomas (Cheung et al., 2008). Consistent with its diverse tumor promoting functions, activated AKT enhances the conversion of the radial to vertical growth phase of melanoma, pointing to its role in progression and metastasis of melanoma (Govindarajan et al., 2007; Fried and Arbiser, 2008).

Despite the fact that a large fraction of melanomas bear mutations in the MAPK signaling pathways, clinical trials using specific MEK and related MAPK inhibitors reveal equal effect on tumors that bear, or not, such mutations. These observations, among others, point to the need to approach melanoma therapy with a combination of targeted therapy. One of the signaling pathways altered in melanoma is the PI3K/AKT signaling pathway. The present study provides initial characterization of an AKT inhibitor, BI-69A11, which inhibits AKT phosphorylation and expression levels. BI-69A11 effectively caused the death of melanoma cells in culture and the regression of melanoma xenografts, thereby justifying further studies for pre-clinical and clinical evaluations. Accordingly, there is a present need to identify and characterize a selective inhibitor of AKT activation for use in tumor therapy.

In addition to Protein Kinase B, Nuclear Factor kappa-light-chain-enhancer of activated B cells (NFkB) is a protein complex involved in many aspects of cellular activity. NFkB is found in almost all animal cell types and is involved in cellular responses to stimuli such as stress, cytokines, free radicals, ultraviolet irradiation, oxidized LDL, and bacterial or viral antigens. Incorrect regulation of NFkB has been linked to cancer, inflammatory and autoimmune diseases, septic shock, viral infection, and improper immune development. NFkB is involved in many aspects of cell growth, differentiation and proliferation via the induction of certain growth and transcription factors (e.g. c-myc, ras and p53).

While in an inactivated state, NFk is located in the cytosol complexed with the inhibitory protein IkBα. Through the intermediacy of integral membrane receptors, a variety of extracellular signals can activate the enzyme IkB kinase (IKK). IKK, in turn, phosphorylates the IkBα protein, which results in ubiquitination, dissociation of IκBα from NFkB, and eventual degradation of IκBα by the proteosome. The activated NFkB is then translocated into the nucleus where it binds to specific sequences of DNA called response elements (RE). The DNA/NFkB complex then recruits other proteins such as coactivators and RNA polymerase, which transcribe downstream DNA into mRNA, which, in turn, is translated into protein, which results in a change of cell function. (Maniatis, 1999, Genes Dev., 13: 505).

After activation of cells by e.g. the binding of certain cytokines to their surface receptors, the IkB proteins are rapidly phosphorylated. Two kinases have been identified, that are responsible for this modification of the IkBs: IKK-alpha and IKK-beta. Both kinases were identified to be members of a high molecular complex which also contains IKK-gamma (also called NEMO, IKKAP) and IKAP. IKK-alpha and IKK-beta share significant sequence homology and contain identical structural domains. By their leucine-zipper domains they form heterodimers, in vivo. (May and Ghosh, seminars in Cancer Biology, 1997, 8: 63-73).

Upon DNA damage eukaryotic cells respond by either cell cycle arrest, activation of DNA repair or cell death. In order to prevent damaged DNA from replicating several checkpoint proteins are induced. Phosphatidylinositol 3-like kinases, ataxia telangiectasia-mutated (ATM) responds to abnormal DNA initiating checkpoints. ATM responds to DNA damaged by ionizing radiation causing a double-strand break in the DNA. A checkpoint protein that has been identified as a substrate for ATM kinase includes Chk2. (Kudoh, et al., 2005, J. Biol. Chem., 280; 8156-8163).

Because of the importance of the AKT pathway in cancer, as well as in other diseases that involve disruption of normal apoptotic processes, including neurodegenerative conditions, there is a need for improved screening methods for the discovery of inhibitors of Akt1, as well as for compounds and compositions discovered by such screening methods. The present invention describes the characterization of the AKT inhibitor BI-69A11 in UACC903 melanoma cells, which harbor a PTEN mutation, and in 29-1, a UACC903 variant that was reconstituted with chromosome 10 carrying a wt PTEN. Additionally, there is a demonstration of the inhibition of AKT activity by BI-69A11 and its effect on melanoma cells in culture and xenograft models. Lastly, BI-69A11 was tested for inhibition of additional protein kinases.

### SUMMARY OF THE INVENTION

One aspect of the invention is a screening method that meets these needs and provides efficient, high throughput screening of compounds for Akt1 inhibitory activity. In general, this screening method comprises:
(1) providing a plurality of compounds suspected of having Akt1 kinase inhibitory activity;
(2) modeling the docking of each of the plurality of the compounds with a target binding site derived from the crystal structure of a ternary complex involving Akt1, a nonhydrolyzable ATP analogue, and a peptide substrate derived from a physiological AKT substrate such that the protein active site is defined including those residues within a defined distance from the nonhydrolyzable ATP analogue;
(3) ranking the docked compounds by goodness of fit;
(4) further selecting compounds from compounds high ranked by goodness of fit in docking by using one or more screening criteria;
(5) optionally, visually analyzing structures of compounds selected in step (4) to remove any compounds with improbable docking geometry; and
(6) experimentally testing the selected compounds from step (4) or step (5), if step (5) is performed, to determine their inhibitory activity against Akt1 in order to select compounds with Akt1 inhibitory activity.

Typically, the nonhydrolyzable ATP analogue is AMP-PNP. Typically, the peptide substrate is a peptide substrate derived from GSK-3β.

Typically, the defined distance from the nonhydrolyzable analogue is from about 6.0 Å to about 7.0 Å. Preferably, the defined distance from the nonhydrolyzable analogue is about 6.5 Å.

Typically, the modeling of docking is performed using a docking algorithm. Preferably, the docking algorithm is FlexX.

Typically, the step of further selecting compounds from compounds high ranked by goodness of fit in docking by using one or more screening criteria is performed by using one or more of CSCORE (SYBYL), Drugscore, Goldscore, Chemscore, and GOLD.

Preferably, when the docking algorithm is FlexX, the step of further selecting compounds from compounds high ranked by goodness of fit in docking by using one or more screening criteria is performed by first using Drugscore, and then evaluating and ranking the top docked structures according to Goldscore and Chemscore individually. More preferably, compounds that are highly ranked according to both Goldscore and Chemscore functions, when those are applied individually, are then selected for visual analysis to remove compounds with improbable docking geometries.

Typically, the step of experimentally testing the compounds that emerge from screening in step (4) or step (5), if performed, is performed by testing the compounds at a concentration up to 30 μM. More typically, the concentration is 10 μM.

Typically, compounds screened as positive are capable of binding specifically within the catalytic site of the ATP. Typically, compounds screened as positive act as competitive inhibitors of Akt1, competing with ATP. Typically, compounds screened as positive are involved in hydrogen-bonding interactions with residues Lys181, Ala232, Thr292, and Thr162 of Akt1.

The method can further comprise an additional screening step of measuring a consensus between scoring patterns and hydrogen bonding patterns substantially similar to that observed in the crystal structure of Akt1 in complex with AMP-PMP and selecting compounds that exhibit both highly ranked scoring patterns and hydrogen bonding patterns substantially similar to that observed in the crystal structure of Akt1 in complex with AMP-PMP.

Another aspect of the invention is a method of derivatizing a compound determined to have inhibitory activity against Akt1 kinase to improve its inhibitory activity comprising the steps of:
(1) providing a compound having inhibitory activity against Akt1 kinase;
(2) derivatizing the compound by introducing at least one covalent modification thereto to produce at least one derivative; and
(3) screening the derivatives produced in step (2) for inhibitory activity against Akt1 kinase; and
(4) selecting a derivative that has improved inhibitory activity against Akt1 kinase as compared with the compound provided in step (1).

The step of derivatizing typically comprises at least one reaction selected from the group consisting of the substitution of halogens for one or more hydrogens; the replacement of halogens by hydrogens; the placement, removal or repositioning of carboxyl groups on aromatic rings; the conversion of carboxylic acids into esters and vice versa; the conversion of alcohols into ethers; the substitution of hydrogens on amine groups with alkyl groups; and the removal of alkyl groups on amine groups.

Another aspect of the invention is a pharmaceutical composition for inhibiting Akt 1 kinase comprising:
(a) a compound whose activity in inhibiting Akt1 kinase was discovered by the screening method of the present invention in a quantity sufficient to inhibit Akt1 kinase; and
(b) a pharmaceutically acceptable carrier.

The compound characterized in the present invention is BI-69A11, a compound which was shown to inhibit AKT activity in in vitro kinase assays. Analysis of BI-69A11 was performed in melanoma cells, a tumor type that commonly exhibits upregulation of AKT. Treatment of the UACC903 human melanoma cells, harboring the PTEN mutation, with BI-69A11 caused efficient inhibition of AKT S473 phosphorylation with concomitant inhibition of AKT substrate PRAS40. Treatment of melanoma cells with BI-69A11 also reduced AKT protein expression, which coincided with inhibition of AKT association with HSP90. BI-69A11 treatment not only caused cell death of melanoma, but also prostate tumor cell lines. Notably, the effect of BI-69A11 on cell death was more pronounced in cells that express an active form of AKT. Significantly, intra-peritoneal injection of BI-69A11 caused effective regression of melanoma tumors xenografts, which coincided with elevated levels of cell death. These findings identify BI-69A11 as a potent inhibitor of AKT that is capable of eliciting effective regression of xenograft melanoma tumors

Another aspect of the present invention relates to compositions having a structure of BI-69A11, wherein the structure is capable of inhibiting AKT activation. Preferably, the structure is used in a preparation for tumor therapy.

Yet, another aspect of the present invention involves a method of decreasing AKT expression based on the presence of a compound with a structure similar to BI-69A11 in proximity to a tumor cell, wherein the tumor cell is one which harbors an active form of AKT.

Another aspect of the invention is the BI-69A11 compound which was shown to inhibit the NFkB pathway in in vitro kinase assays. Analysis of BI-69A11 was performed in melanoma cells. Treatment of the UACC903 human melanoma cells, harboring the PTEN mutation, with BI-69A11 caused efficient inhibition of the NFkB pathway.

Another aspect of the present invention relates to compositions having a structure of BI-69A11, wherein the structure is capable of inhibiting NFkB pathway. Preferably, the structure is used in a preparation for tumor therapy.

In yet another aspect of the invention the BI-69A11 compound was shown to activate ATM-Chk signaling activating DNA damage response.

Another aspect of the invention relates to compositions having a structure of BI-69A11, wherein the structure is capable of activating the ATM-Chk signaling activating DNA damage response. Preferably, the structure is used in preparation for tumor therapy.

Yet another aspect of the present invention provides for compounds and methods of inhibiting tumor growth by administration to a patient having melanoma cancer an amount of BI-69A11 sufficient to reduce tumor size.

Yet another aspect of the present invention provides for targeting specific isoforms of AKT by administering a compound specific for inhibiting protein expression of an AKT isoform to an animal having a melanoma, wherein the melanoma exhibits an active form of AKT and the compound is structurally similar to BI-69A11.

Yet another aspect of the invention is a method of treating a disease or condition characterized by dysregulation of apoptosis comprising administering an effective quantity of the pharmaceutical composition according to the present invention to a subject diagnosed with or suspected of having a disease or condition characterized by dysregulation of apoptosis in order to normalize apoptosis. The disease or condition can be cancer or another condition, such as a neurodegenerative condition.

Yet another aspect of the invention is a method of preventing cancer comprising administering a therapeutically effective amount of BI-69A11 to a patient.

### BRIEF DESCRIPTION OF THE DRAWINGS

The following invention will become better understood with reference to the specification, appended claims, and accompanying drawings, where:

FIG. 1 is a schematic representation of the virtual docking approaches adopted: (A) an approach involving docking of 50,000 compounds and ranking according to the software FlexX, then ranking the top scoring 2000 compounds with other scoring functions using CSCORE, as well as selecting top ranking compounds with Drugscore, Goldscore, and Chemscore, as well as docking the FlexX top 4000 compounds using GOLD; followed by experimental testing; (B) an approach selecting the top 4000 compounds out of 50,000 docket compounds using FlexX and Drugscore; the top 4000 docked structures were then evaluated and ranked according to Goldscore and Chemscore functions (CSCORE); a list of common 200 compounds was then selected among ranked top 700 compounds according to both scoring functions, and elimination of structures with improbable docking geometry by visual analysis, followed by experimental testing of the remaining 100 compounds.

FIG. 2 is a series of graphs showing the assay of Akt1 inhibition for Compounds 1 and 2: (A) IC₅₀ evaluation for Compound 1; (B) IC₅₀ evaluation for Compound 2; (C) Lineweaver-Burk Km and Km(app) evaluation for Akt1; (D) Akt1 inhibition assay using GSK-3 as a substrate, showing a comparison of Compound 1 and Compound 2 with H89 at 10 μM using an immunological approach after polyacrylamide gel electrophoresis and transfer to a nitrocellulose membrane with rabbit polyclonal anti-phospho-GSK-3α/β (Ser21/9); and (E) dose response for Compound 1.

FIG. 3 shows docking models: (A-C), docked structures of Compounds 1-3 into the ATP binding site of Akt1; (D) hydrogen bonds between Compound 1 and amino acid residues present in the Akt1 catalytic pocket.

FIG. 4 depicts the predicted binding mode of BI-69A11 in the ATP site of PKB/AKT. Hydrogen bonds are denoted by dashed cylinders in yellow.

FIG. 5 shows the effect of BI-69A11 on melanoma and prostate cancer cells. (A) MeWo melanoma cells growing in 60 mm plates were treated with the indicated concentration of the inhibitor for 4 h before proteins were prepared for western blot analysis using the indicated antibodies. Level of beta.actin was monitored as a control for protein loading. (B) Experiment was performed as indicated in panel A, except that cells were harvested after 24 h for analysis of cell death using trypan blue staining. (C) Melanoma (MeWo), prostate cancer (PC3) and breast cancer (MCF7) cells were treated with BI-69A11 at the indicated concentrations; levels of AKT phosphorylation or expression were assessed 4 h later. The level of beta.actin used as a control for protein loading. (D) Experiment was performed as indicated in panel C, except that cells were harvested after 4 h for analysis of cell death using trypan blue staining.

FIG. 6 describes the effect of BI-69A11 on phosphorylation and total levels of AKT in UACC 903 (PTEN−) cells. (A) UACC903 and the modified clone 29-1 (PTEN+) cells were exposed to 10 micro.M BI-69A11 and 50 micro.M LY294002 (a PI3K inhibitor) and the levels of pAKT473 and total AKT determined by western analysis. Reduced levels of pAKT473 were observed in response to both BI-69A11 and LY, and reduced levels of total AKT were observed with increasing time after treatment with BI-69A11 in 903 and 29-1 cells. (SF=Serum Free). (B) BI-69A11 inhibits PRAS40 phosphorylation. UACC903 melanoma cells or 29-1 cells (PTEN+) were subjected to treatment with IGF-1 (20 ng/mL) and the level of PRAS40 phosphorylation was assessed. As indicated in FIG. 3B, cells were also treated with BI-69A11 (10.micro.M). The cells were collected, lysed and levels of pPRAS40 and total PRAS40 determined by western analysis. (C) AKT association with HSP90 is inhibited by BI-69A11. Human melanoma cells were treated with BI-69A11 (10.micro.M) or DMSO control for 4 h, followed by protein preparation and immunoprecipitation using antibodies to pan-AKT. MG132 treatment, when used, initiated 1 h prior to treatment and lasted up to the preparation of proteins (total of 5 h). The right panel depicts analysis of total lysates.

FIG. 7 demonstrates BI-69A11 elicits more efficient cell death in UACC903 cells, compared with 29-1 cells. (A) 903 and 29-1 cells were exposed to different concentrations of BI-69A11 or UV light as a positive control and the percentage of cell death determined by trypan blue exclusion assay. (B) PARP cleavage was determined in 903 and 29-1 cells after treatment with BI-69A11 at 2, 4 and 6 hours. Increased PARP cleavage is observed in 903 cells as compared to that observed in 29-1 cells after treatment with BI-69A11.

FIG. 8 shows BI-69A11 causes regression of melanoma tumors. (A) Nude mice were subcutaneously injected with UACC 903 cells and tumors were allowed to reach an approximate size of 1 mm.sup.3 before intraperitoneal treatment was started; treatment was performed twice per week for 3 weeks with the indicated concentrations of BI-69A11. Each of the experimental groups consisted of 10 mice that were subjected to treatment with the indicated concentration of BI-69A11. Half of the animals were injected with tumor cells, and half with the control solution. Tumors were measured with calipers at the indicated time points. The mice were sacrificed and tumors harvested at the end of this period were measured and weighed to determine tumor volume and mass. Blue bars represent control-vehicle treated group; yellow bars represent the 5 mg/Kg treatment group; the orange and green bars represent the 1 and 2 mg/Kg treatment groups, respectively. (B) Analysis of cell death in the tumors was performed using Tunel staining. Shown are representative pictures for control tumors (right) and a treatment group (5 mg/Kg; left). (C) Quantification of Tunel staining in the different tumor groups. Samples from each of the tumors were subjected to Tunel staining which was quantified by Aperio ImageScope and confirmed by visual inspection of representative samples. The percentage of Tunel positive cells is indicated in the graph. Blue bars represent control-vehicle treated group; yellow bars represent the 5 mg/Kg treatment group; the orange and green bars represent the 1 and 2 mg/Kg treatment groups, respectively.

FIG. 9 depicts the synthesis of (E)-3-(3-(1H-benzo[d]imidazol-2-yl)acryloyl)-6-chloro-4-phenylquinolin-2(1H)-one (4, BI-69A11). The compound was synthesized from (2-amino-5-chlorophenyl)-(phenyl)-methanone (1) through the Friedlander condensation reaction according to the reported procedures (De and Gibbs, 2005). To a solution of (3) (511 mg, 1.72 mmol) in ethanol (8 mL), a 20% NaOH solution in water (1.3 mL) was added at room temperature. After stirring for 15 min, 1H-benzoimidazole-2-carboxaldehyde (376 mg, 2.58 mmol) was added to the reaction mixture and the resulting reaction mixture was stirred for 16 hours at room temperature. Just before the completion of the reaction, it was neutralized with 1N HCl, yielding a precipitate that was collected and purified by flash chromatography (60 to 80% ethyl acetate in hexane) to result in the final compound in 50% yield (330 mg).

FIG. 10 is shows the effect of BI-69A11 inhibiting the NFkB pathway. UACC903 melanoma cells were exposed to 10 μM BI-69A11 or vehicle control for 1 h prior to treatment with 20 ng/ml TNF-α. Cells were harvested at the indicated time points and whole cell lysates were subjected to western analysis for phosphorylated IKK, total IKK, phosphorylated IkB, total IkB, and PLCγ (as loading control).

FIG. 11 describes how selected BI-69A11 analogs inhibit the NFkB pathway. UACC903 melanoma cells were exposed to vehicle control, 10 μM BI-69A11, or 10 μM analog for 1 h prior to stimulation with 20 ng/ml TNF-α as indicated. Cells were harvested at 5 min post-stimulation and whole cell lysates were subjected to western analysis for phosphorylated IKK, total IKK, phosphorylated IkB, total IkB, and PLC7 (as loading control).

FIG. 12 shows decreased cell viability in the presence of BI-69A11 or the analog BI-83G10. UACC903 melanoma cells were treated with vehicle (0 μM) or the indicated concentrations of BI-69A11, BI-83G10, or BI-98C11. 24 h post-treatment, cell viability was determined using the CellTiter Blue Cell Viability Assay (Promega, Madison Wis.) following the manufacturer's directions.

FIG. 13 shows how BI-69A11 activates the DNA damage response. UACC903 melanoma cells were exposed to vehicle control or 10 μM BI-69A11 for 1 h prior to γ-irradiation (10 Gray). Cells were harvested at the indicated time points post-irradiation and whole cell lysates were subjected to western analysis for phosphorylated ATM, total ATM, phosphorylated Chk2, and PLCγ (as loading control).

FIG. 14 is an assessment of BI-69A11 analogs on Chk2 phosphorylation. UACC903 melanoma cells were exposed to vehicle control, 10 μM BI-69A11, or 10 μM analog for 2 h. Cells were harvested and whole cell lysates were subjected to western analysis for phosphorylated Chk2 and PLCγ (as loading control).

FIG. 15 shows the analogs of BI-69A11 including, BI-83G10, BI-98C11, BI-103F6, BI-83H2, BI101G9, BI-87A3.

FIG. 16A is a graph of the number of tumors vs. weeks since induction of mutant N-Ras expression in NRAS/INK4a mice.

FIG. 16B is a graph of the numbers of melanomas and fibrosarcomas in NRAS/Ink4a mice.

### DETAILED DESCRIPTION OF THE INVENTION

One aspect of the present invention provides compounds and methods of preventing the development of melanoma by administering to a patient an amount of BI-69A11 sufficient to inhibit development of melanoma. In one aspect, the compound is administered to a subject having or at risk of having melanoma prior to showing symptoms of melanoma. In another aspect, the compound is administered to a subject having pre-cancerous lesions but prior to development of melanoma. The compound can be administered alone or in combination with other anti-cancer compounds.

One aspect of the invention is a method of screening compounds for inhibition of Akt1 kinase activity comprising the steps of
(1) providing a plurality of compounds suspected of having Akt1 kinase inhibitory activity;
(2) modeling the docking of each of the plurality of the compounds with a target binding site derived from the crystal structure of a ternary complex involving Akt1, a nonhydrolyzable ATP analogue, and a peptide substrate derived from a physiological AKT substrate such that the protein active site is defined including those residues within a defined distance from the nonhydrolyzable ATP analogue;
(3) ranking the docked compounds by goodness of fit;
(4) further selecting compounds from compounds high ranked by goodness of fit in docking by using one or more screening criteria; and
(5) optionally, visually analyzing structures of compounds selected in step (4) to remove any compounds with improbable docking geometry; and
(6) experimentally testing the selected compounds from step (4) or step (5), if step (5) is performed, to determine their inhibitory activity against Akt1 in order to select compounds with Akt1 inhibitory activity.

Typically, the nonhydrolyzable ATP analogue is AMP-PNP. Typically, the peptide substrate is a peptide substrate derived from GSK-3β.

Typically, the defined distance from the nonhydrolyzable analogue is from about 6.0 Å to about 7.0 Å. Preferably, the defined distance from the nonhydrolyzable analogue is about 6.5 Å.

Typically, the modeling of docking is performed using a docking algorithm. A particularly preferred docking algorithm is FlexX (BiosolvelT, Sankt Augustin, Germany), but others are known in the art.

The step of further selecting compounds from compounds high ranked by goodness of fit in docking by using one or more screening criteria can employ various screening criteria known in the art, or combinations of those screening criteria. For example, screening can be accomplished using CSCORE (SYBYL) (14), Drugscore (15), Goldscore (16), Chemscore (17), or GOLD (18). These screening methods can be applied sequentially, so that compounds that are high ranked by one screening method can then be rescreened with a second method, and compounds ranked high in both screening methods are selected for further analysis. In one particularly preferred approach, compounds are selected using FlexX and Drugscore, and the top docked structures are evaluated and ranked according to Goldscore and Chemscore functions individually. Compounds that are highly ranked according to both Goldscore and Chemscore functions, when those are applied individually, are then selected for visual analysis to remove compounds with improbable docking geometries.

The molecular parameters that govern binding of substrates and inhibitors to enzymes are well known in the art. Typically, binding is governed by hydrogen bonding, hydrophobic interactions, ionic bonds (salt links), covalent bonds (at certain stages of the reaction), and Van der Waals forces; binding typically involves either a “lock and key” mechanism or an “induced fit” mechanism. These can be modeled by means of appropriate software, taking into account the variation in the strength of the interaction with the distance between the two molecules and that there are six degrees of rotational and translational freedom of one molecule relative to the other as well as the conformational degrees of freedom of each molecule.

Typically, the step of experimentally testing the compounds that emerge from screening in step (4) or step (5), if applicable, are tested at 10 μM or at concentrations up to 30 μM for their Akt1 inhibitory activity. Typically, inhibitory activity is evaluated for the selected compounds by using the Z'-LYTE kit assay provided by Invitrogen Corporation (19).

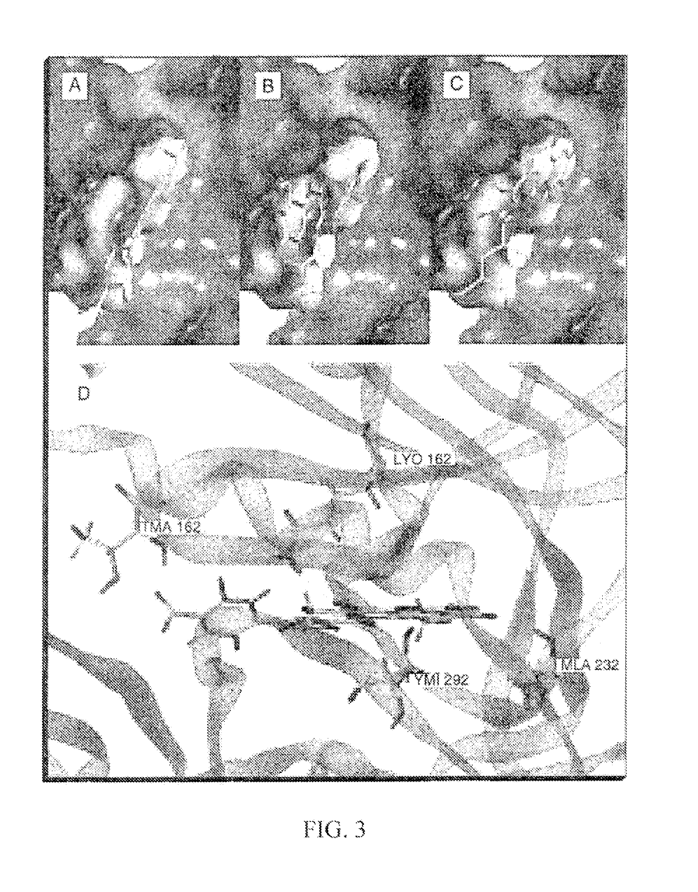

Typically, compounds screened as positive are capable of binding specifically within the catalytic site of the ATP, resembling the binding of the adenosine moiety of this cofactor (FIGS. 3A-C). Kinetic analysis establishes that these compounds act as typical competitive inhibitors; they compete with ATP for binding by the kinase. Accordingly, they affect the Kₘ rather than the Vₘₐₓ, of the kinase reaction. Competitive inhibition is well-understood in enzymology, and the consequences of competitive inhibition need not be recited herein. Typically, compounds screened as positive are involved in hydrogen-bonding interactions with residues Lys181, Ala232, Thr292, and Thr162 (FIG. 3D) similar to interactions observed in the crystal structure of Akt1 in complex with AMP-PNP. Therefore, in one preferred alternative, another screening step is performed, that of measuring a consensus between scoring patterns and hydrogen bonding patterns substantially similar to that observed in the crystal structure of Akt1 in complex with AMP-PMP and selecting compounds that exhibit both highly ranked scoring patterns and hydrogen bonding patterns substantially similar to that observed in the crystal structure of Akt1 in complex with AMP-PMP. This measurement of the consensus improves the hit rate of the overall screening process substantially.

The compounds to be selected can be from any suitable library of small molecule compounds. One library is obtainable from Chembridge (San Diego, Calif.). Other libraries are available, and methods for their preparation are described, for example, in R. B. Silverman, “The Organic Chemistry of Drug Design and Drug Action” (2d ed., Elsevier. Amsterdam), pp. 41-43, incorporated herein by this reference. Scaffolds for synthesis can be derived, for example, from natural products.

Among the compounds having Akt1 inhibitory activity are Compounds 1 and 2 (Table 1), showing IC₅₀ values in the low-micromolar range. Compound 3 had an IC₅₀ of 25.1 μM.

In addition, Compounds 4 and 5 (Table 2), which are derivatives of Compound 1, have limited inhibitory activity against Akt1 kinase.

### DEFINITIONS

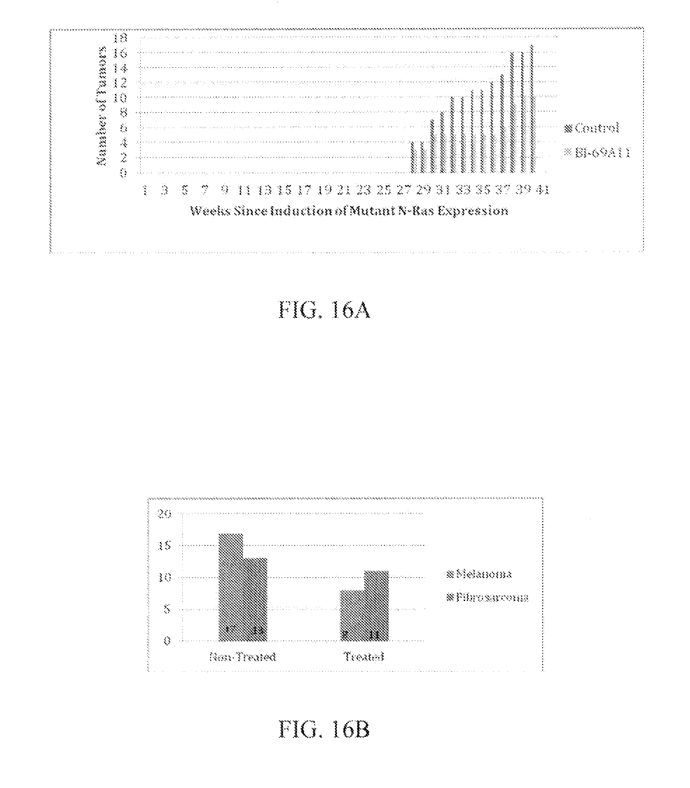

When referring to BI-69A11 the term “analogs” or “derivitives” includes but is not limited to the formulas shown in FIG. 16 including, BI-83G10, BI-98C11, BI-103F6, BI-83H2, BI-101G9, BI-87A3.

The compound BI-69A11 and its analogs contain one or more chiral centers, and exist in different optically active forms. When compounds contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound has one or more chiral substituent it may exist in diastereoisomeric forms. The diastereoisomeric pairs may be separated by methods known to those skilled in the art, for example chromatography or crystallization and the individual enantiomers within each pair may be separated as described above. The present invention includes each diastereoisomer of compounds of Structural Formula I and mixtures thereof.

Certain compounds of BI-69A11 may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of BI-69A11 and mixtures thereof.

Certain compounds of BI-69A11 may exist in zwitterionic form and the present invention includes each zwitterionic form of compounds of BI-69A11 and mixtures thereof.

Unless otherwise indicated, all anhydrous solvents were commercially obtained and stored in Sure-seal bottles under nitrogen. All other reagents and solvents were purchased as the highest grade available and used without further purification. NMR spectra were recorded on Varian 300 or 500 MHz instruments. Chemical shifts ( ) are reported in parts per million (ppm) referenced to 1H (Me4Si at 0.00). Coupling constants (J) are reported in Hz throughout. Mass spectral data were acquired on an Esquire LC00066 for low resolution, a Micromass 70 SEQ for high resolution, or a JEOL LC-mate tuned for either low resolution or high resolution. The progress of reactions was monitored by TLC.

The term “pharmaceutically acceptable” means that the carrier, diluent, excipients and salt must be compatible with the other ingredients of the formulation, and not deleterious to the recipient thereof. Pharmaceutical formulations of the present invention are prepared by procedures known in the art using well known and readily available ingredients.

“Effective amount” means an amount of compound according to Structural Formula I, in any polymorphic form, or a salt thereof that is capable of producing its intended effect.

The language a “therapeutically effective amount” or “pharmaceutically effective amount” is intended to include an amount which is sufficient to mediate a disease or condition and prevent its further progression or ameliorate the symptoms associated with the disease or condition. Such an amount can be administered prophylactically to a patient thought to be susceptible to development of a disease or condition. Such amount when administered prophylactically to a patient can also be effective to prevent or lessen the severity of the mediated condition.

“Preventing” refers to reducing the likelihood that the recipient will incur or develop any of the pathological conditions described herein.

The term “cell lines”, as used herein, unless otherwise indicated, refer to UACC 903 cells and 29-1 cells. The cells were cultured in DMEM, supplemented with 10% FBS and maintained at 37° C. in 5% CO2. Cells were plated to 75% confluency and grown overnight at 37° C. Plates were rinsed twice with a serum-free DMEM medium and the appropriate concentration of the compound was added to the plates. Cells were harvested at different timepoints by scraping (for western blotting) or trypsinization (for cell proliferation assay) where appropriate.

Accordingly, another aspect of the present invention is a method of derivatizing a compound determined to have inhibitory activity against Akt1 kinase to improve its inhibitory activity comprising the steps of:
(1) providing a compound having inhibitory activity against Akt1 kinase;
(2) derivatizing the compound by introducing at least one covalent modification thereto to produce at least one derivative; and
(3) screening the derivatives produced in step (2) for inhibitory activity against Akt1 kinase; and
(4) selecting a derivative that has improved inhibitory activity against Akt1 kinase as compared with the compound provided in step (1).

The derivatization can include one or more reactions well known in organic chemistry and in the art of drug design, including the substitution of halogens for one or more hydrogens and the replacement of halogens by hydrogens, the placement, removal or repositioning of carboxyl groups on aromatic rings, the conversion of carboxylic acids into esters and vice versa, the conversion of alcohols into ethers, the substitution of hydrogens on amine groups with alkyl groups or the removal of alkyl groups on amine groups, and other similar reactions. The derivatization can be carried out under standard reaction conditions employing reagents well known in the art, such as those disclosed in M. B. Smith & J. March, “March's Advanced Organic Chemistry: Reactions, Mechanisms, and Structure (5^{th} ed., John Wiley & Sons, New York, 2001), incorporated herein by this reference. Other derivatization reactions can be used.

Accordingly, another aspect of the invention is a pharmaceutical composition for inhibiting Akt1 kinase comprising:
(1) a compound whose activity in inhibiting Akt1 kinase was discovered by the screening method described above in a quantity sufficient to inhibit Akt1 kinase; and
(2) a pharmaceutically acceptable carrier.

Typically, the compound has an IC₅₀ of less than about 100 μM. Preferably, the compound has an IC₅₀ of less than about 30 μM. More preferably, the compound has an IC₅₀ of less than about 10 μM. Still more preferably, the compound has an IC₅₀ of less than about 5 μM.

The pharmaceutical composition can be formulated for the treatment of cancer or for the treatment of another condition characterized by dysregulation of apoptosis, including neurodegenerative conditions.

Among preferred compounds for the preparation of pharmaceutical compositions are Compounds 1, 2, 3, 4, and 5. Among particularly preferred compounds for the preparation of pharmaceutical compositions are Compounds 1 and 2, so that the compound is selected from the group consisting of Compound 1 of formula (I), Compound 2 of formula (II), Compound 3 of formula (III), and Compounds 4 and 5 of formula (IV), where, in formula IV, for Compound 4, R is p-COOH and for Compound 5, R is m-COOH

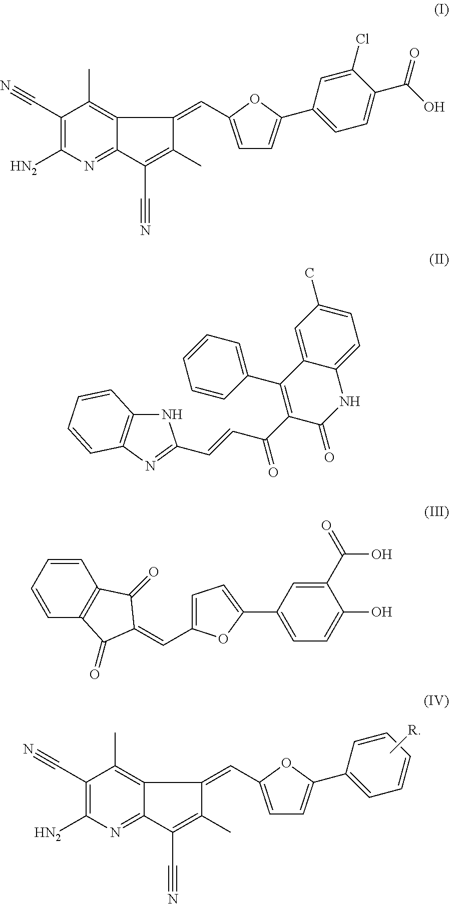

Toxicity and therapeutic efficacy of compounds in pharmaceutical compositions according to the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

For any compound used in the pharmaceutical compounds according to the present invention, the therapeutically effective dose can be estimated initially from cell culture assays. For example, a dose can be formulated in animal models to achieve a circulating plasma concentration range that includes the IC₅₀ as determined in cell culture (i.e., the concentration of the test compound which achieves a half-maximal improvement in receptor signaling when chronic effects are considered). Such information can be used to more accurately determine useful doses in humans. Levels in plasma may be measured, for example, by HPLC.

The exact formulation, route of administration and dosage for pharmaceutical compositions according to the present invention can be chosen by the individual physician in view of the patient's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps the dose frequency, will also vary according to the age, body weight, and response of the individual patient. A program comparable to that discussed above may be used in veterinary medicine.

Depending on the specific conditions being treated, such pharmaceutical compositions may be formulated and administered systemically or locally. Typically, administration is systemic. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few. Typically, oral administration is preferred.

For injection, the pharmaceutical compositions of the invention may be formulated in aqueous solutions. For such transmucosal administration, penetrants appropriate to the barrier to be permeated are used in the formulation. Such penetrants are generally known in the art.

Use of pharmaceutically acceptable carriers is within the scope of the invention. For example, for oral administration, carriers are well known in the art. Such carriers enable the compounds of the invention to be formulated as tablets, pills, capsules, liquids, gels, syrups, slurries, suspensions and the like, for oral ingestion by a patient to be treated.

In addition to the active ingredients, such as the compound with Akt1 kinase inhibition activity, these pharmaceutical compositions may contain suitable pharmaceutically acceptable carriers comprising excipients and auxiliaries which facilitate processing of the active compounds into preparations which can be used pharmaceutically.

The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methylcellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

TABLE 1
Structures of Compounds Showing Inhibitory Activity Against Akt1

| Molecule | ID | Structure | IC₅₀ (μM) | Ki (μM) |
|---|---|---|---|---|
| 1 | 6025233 | 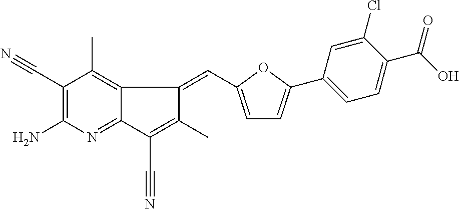 | 2.6 | 1.1 |
| 2 | 5809365 | 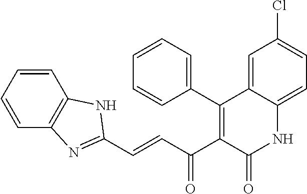 | 4.5 | 3.9 |
| 3 | 5378650 | 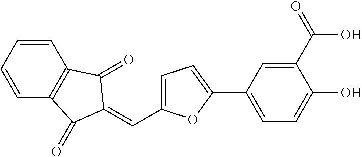 | 25.1 | 20.8 |

TABLE 2
Derivatives of Compound 1 and Their Inhibitory Activity Against Akt1

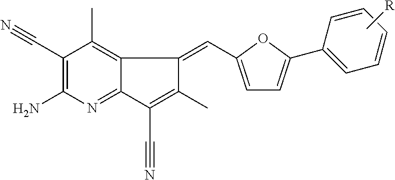

| Compound | R | IC₅₀ | |
|---|---|---|---|
| 1 | para-COOH, meta-Cl | 2.6 | μM |
| 4 | para-COOH | 126 | μM |
| 5 | meta-COOH | 60.2 | μM |

Another aspect of the present invention relates to compositions having a structure of BI-69A11, wherein the structure is capable of inhibiting AKT activation. Preferably, the structure is used in a preparation for tumor therapy.

The term “BI-69A11”, as used herein, refers to Compound 2 with a chemical structure of:

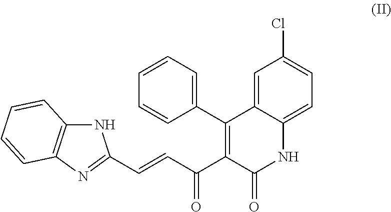

and was synthesized and purified as described herein. It was dissolved in DMSO and maintained as a 100 mM stock.

Yet, another aspect of the present invention involves a method of decreasing AKT expression based on the presence of a compound with a structure similar to BI-69A11 in proximity to a tumor cell, wherein the tumor cell is one which harbors an active form of AKT.

Yet another aspect of the present invention provides for compounds and methods of inhibiting tumor growth by administration to a patient having melanoma cancer an amount of BI-69A11 sufficient to reduce tumor size.

Yet another aspect of the present invention provides for targeting specific isoforms of AKT by administering a compound specific for inhibiting protein expression of an AKT isoform to an animal having a melanoma, wherein the melanoma exhibits an active form of AKT and the compound is structurally similar to BI-69A11.

Another aspect of the invention is the BI-69A11 compound which was shown to inhibit the NFkB pathway in in vitro kinase assays. Analysis of BI-69A11 was performed in melanoma cells. Treatment of the UACC903 human melanoma cells, harboring the PTEN mutation, with BI-69A11 caused efficient inhibition of the NFkB pathway.

Another aspect of the present invention relates to compositions having a structure of BI-69A11, wherein the structure is capable of inhibiting NFkB pathway. Preferably, the structure is used in a preparation for tumor therapy.

In addition to its inhibition of AKT, BI-69A11 was tested for inhibition of additional protein kinases using the Invitrogen SelectScreen Kinase Profiling Service. Nuclear Factor (NF)-kB transcription factors regulate a large set of genes involved in important cellular processes such as stress response, apoptosis, proliferation, immunity, inflammation, and cell adhesion. NF-kB transcription factors are held in an inactive state in the cytoplasm by the inhibitor of NF-kB protein, IkB. The activation of the pathway in response to physiological stimuli requires the phosphorylation and subsequent degradation of IkB proteins, allowing for the nuclear translocation of NF-kB and transcription of target genes. The phosphorylation of IkB proteins is catalyzed by IkB kinases (IKKs) at two amino acid residues (Ser32, Ser36), which in turn promotes degradation of IkB by the ubiquitin-proteasome system.

In yet another aspect of the invention the BI-69A11 compound was shown to activate ATM-Chk signaling activating DNA damage response.

Another aspect of the invention relates to compositions having a structure of BI-69A11, wherein the structure is capable of activating the ATM-Chk signaling activating DNA damage response. Preferably, the structure is used in preparation for tumor therapy.

In addition to its inhibition of AKT, BI-69A11 was tested for inhibition of additional protein kinases using the Invitrogen SelectScreen Kinase Profiling Service. The Ser/Thr protein kinase, Checkpoint Kinase 2 (CHK2), is an important component in the cellular response to DNA damage. In response to genotoxic stresses such as g-irradiation, which causes double stranded DNA breaks, the Ataxia Telangiectasia Mutated (ATM) protein kinase is activated, which in turn phosphorylates a number of downstream effectors, such as CHK2. Activation of the ATM-CHK2 axis enforces the DNA damage checkpoint response, halting the cell cycle in order to initiate DNA repair.

Yet another aspect of the present invention provides for compounds and methods of inhibiting tumor growth by administration to a patient having melanoma cancer an amount of BI-69A11 sufficient to reduce tumor size.

Accordingly, another aspect of the invention is a method of treating a disease or condition characterized by dysregulation of apoptosis comprising administering an effective quantity of a pharmaceutical composition according to the present invention to a subject diagnosed with or suspected of having a disease or condition characterized by dysregulation of apoptosis in order to normalize apoptosis. The disease or condition is typically cancer, but can be a neurodegenerative condition. The subject diagnosed with or suspected of having the disease or condition can be human, but, alternatively, can be a socially or economically important animal selected from the group consisting of a dog, a cat, a sheep, a horse, a cow, a pig, a goat, a chicken, a turkey, a duck, a goose, and any other eukaryote. Apoptosis is a universal process in cell regulation of eukaryotes.

A number of aspects of the invention have been described. Nevertheless, it will be understood that various modifications may be made without departing from the spirit and scope of the invention. Accordingly, the following examples are intended to illustrate but not limit the scope of the invention.

### Example 1

### Selecting Akt1 Inhibitors

Nowadays, high-throughput screening of large chemical databases is a common approach for lead identification. However given the 3D structure of the protein target, it should be possible to restrict the number of compounds to be tested by using computational docking studies.

In this Example, we describe a number of approaches based on the reported crystal structure of Akt1 kinase. This methodology allowed us to select several potential inhibitors on the basis of their predicted ability of docking into the ATP binding site.

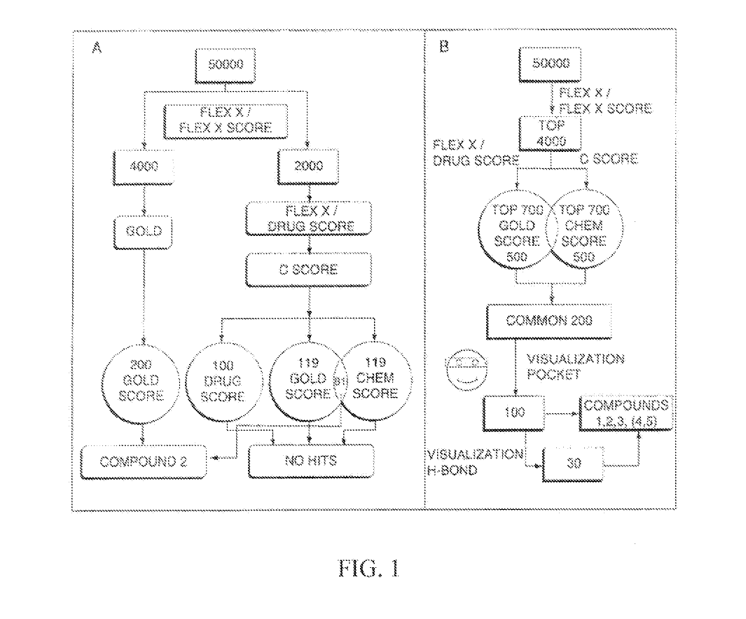

A target binding site was derived from the crystal structure of the ternary complex involving Akt1, non-hydrolyzable form of ATP (AMP-PNP pdb id: 106K) and the peptide-substrate derived from GSK-3β (10). The protein active site was defined including those residues within 6.5 Å from the ATP mimic. Hydrogen atoms were calculated using Sybyl (11) ((Tripos, St. Louis, Mo.) and water molecules, peptide substrate as well as the ATP mimic were eliminated. 50000 compounds (Chembridge San Diego, Calif., USA) were subsequently docked and ranked according to the software FlexX (BioSolveIT, Sankt Augustin, Germany) (12, 13). In an initial attempt, we selected the top 2000 compounds and ranked them with other scoring functions using CSCORE (14) (Sybyl). Subsequently, we experimentally tested at 10 μM the top 100 compounds according to Drugscore (15), the top 200 compounds according to Goldscore (16) and another top 200 compounds according to Chemscore (17). Disappointingly, only one inhibitor (compound 2, 5809365) common in Goldscore and Chemscore selection was found through the Akt1 assay (Table 1), while no inhibitor was found among compounds selected by Drugscore. In addition, we also docked the FlexX top 4000 compounds using GOLD (18) and subsequently selected and tested the top 200 compounds. Once again, compound 2 resulted as the only inhibitor (FIG. 1A). FIG. 1 shows a schematic representation of the virtual docking approaches adopted.

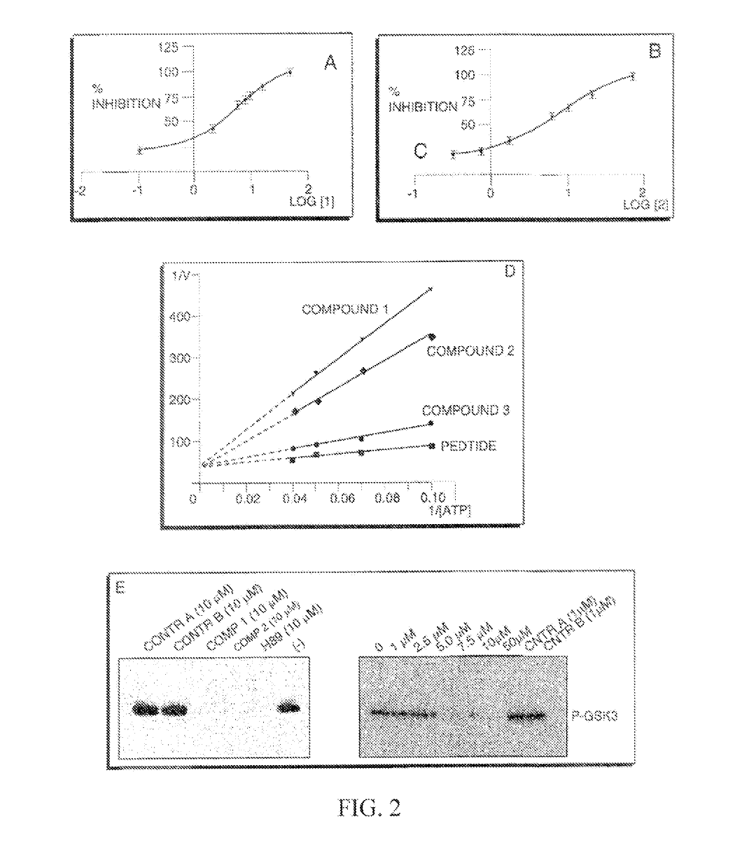

Based on these results, we relied on another strategy described in FIG. 1B. Here the top 4000 compounds out of 50000 docked compounds were selected using FlexX and Drugscore (BioSolvIT). The top 4000 docked structures were further evaluated and ranked according to Goldscore and Chemscore functions (CSCORE). A list of common 200 compounds was then selected among ranked top 700 compounds according to both scoring functions (FIG. 1B). Visual analysis of the 200 docked structures resulted in the elimination of 100 compounds with improbable docking geometry. The remaining 100 compounds were experimentally tested up to 30 μM against Akt1. The inhibitory activity was evaluated for the selected compounds by using Z'-LYTE™ kit assay provided by Invitrogen Corporation (19). Among the experimentally tested compounds at least three emerged as interesting inhibitors, two of which showing IC₅₀ values in the low micromolar range. Particularly, compounds 1 and 2 (Table 1) inhibited Akt1 in a concentration range comparable to that of H-89, the only known commercially available Akt inhibitor (20), yielding IC₅₀ values of 2.3 μM and 4.5 μM, respectively (FIG. 2 A-B). Compound 3 showed an IC₅₀ value of 25.1 μM. Remaining selected compounds did not show any inhibitory activity up to 30 μM. In FIG. 2: A) IC₅₀ evaluation for compound 1 (2.6 μM). The Hill slope for this curve is 1.1; B) IC₅₀ evaluation for compound 2 (4.5 μM). Corning® 384-well low volume plates (20 μl) were used. The fluorescent-enzymatic assay has been performed following the protocol provided by Invitrogen Corporation, using a fluorescent plate reader (Victor2, Perkin-Elmer). IC₅₀ values were determined fitting the data to the sigmoidal dose/response equation and plotting the observed percentage of inhibition versus the logarithm of inhibitor concentration using GraphPad Prism®. C) Lineweaver-Burk Kₘ and Kₘ(app) evaluation for Akt1. Each measurement was performed in triplicate. The Kₘ and Vₘₐₓ values of the enzymatic reaction were determined at 25° C. by using increasing ATP concentrations (5, 10, 15, 20 and 25 μM). The Kᵢ and the Kₘ₍ₐₚₚ₎ were calculated at fixed inhibitor concentrations, as reported in the text. All constant values were definitely evaluated by fitting the data to the Lineweaver-Burk plot; D) Akt1 inhibition assay using GSK-3 as a substrate. Comparison of compounds 1 and 2 (Table 1) with H89 at 10 μM. E) Dose response for compound 1. Akt (10 ng of recombinant enzyme) in 25 μl 1× kinase buffer (25 mM Tris, pH 7.5; 5 mM n-glycerol phosphate; 2 mM dithiothreitol; 0.1 mM Na₃VO₄; and 10 mM MgCl₂), was mixed with 2.5 μl DMSO (1% stock) or MPA-D (100 μM in 1% DMSO). Samples were incubated on ice for 1.5 hours at which time 1 μg of GSK-3 fusion protein (Cell Signaling), which served as the substrate, was added followed by ATP (200 μM) to each reaction mixture. After the suspensions were incubated at 30° C. for 20 min, the reaction was terminated by the addition of 3×SDS sample buffer (187.5 mM Tris-HCl, pH 6.8; 6% SDS; 30% glycerol; 150 mM dithiothreitol; and 0.03% bromophenol blue). The samples were boiled for 5 min, and the proteins were separated on a 12% SDS polyacrylamide gel and subsequently transferred to a nitrocellulose membrane. Membranes were incubated with rabbit polyclonal anti-phospho-GSK-3α/β (Ser21/9) (Cell Signaling).

A second assay was carried out, in order to further evaluate the inhibitory activity for compounds 1 and 2 by using an immuno-blotting assay with anti-phospho-GSK-3α/β and GSK-3 as a substrate (FIG. 2 D-E). In agreement with the Z'-LYTE™ assay, both compounds inhibited GS3K phosphorylation in the low micromolar range.

To confirm and extend these findings, we measured the Kᵢ value and the type of inhibition of Akt1 by compounds 1, 2 and 3 (FIG. 2C). For these purposes, we initially determined the Kₘ and the Vₘₐₓ of the enzymatic reaction involving the peptide provided by the Z'-LYTE™ kit assay and Akt1 by varying the concentration of ATP. The above parameters appeared to be 7.9 μM and 0.0205 μmol min⁻¹ mg⁻¹, respectively. We then used a 10 μM concentration of compound 1, 20 μM of compound 2 and 50 μM of compound 3, in order to identify the inhibitors' Kᵢ values (Table 1). Because all our inhibitors affected the Kₘ rather than the Vₘₐₓ of the reaction (FIG. 2C), they can be truly considered ATP-competitive inhibitors of Akt1.

To rule out the possibility of eventual non-specific interactions, we also verified that no substantial changes in the IC₅₀ values for compounds 1 were detected, when increasing 10 fold the protein concentration as well as by pre-incubating the compounds with Akt1 for 30 minutes prior measuring its IC₅₀ value. These simple tests have been shown to give dramatically different IC₅₀ values in presence of non-specific ligand-protein interactions (21).

In addition, we tested our compounds against a non related protein kinase, Abl1 (22), which is a tyrosine kinase under investigation in our laboratory. Our compounds did not inhibit this kinase at concentrations up to 100 μM. We currently do not have data on the selectivity of our compounds for the different Akt isoforms.

Therefore using our structure-based approach we were able to identify three inhibitors of Akt1 (Table 1), two of which showed an inhibitory activity comparable to that of H-89 (FIG. 2). Based on the docked geometry, and in agreement with our experimental data, it appears that all three inhibitors place themselves nicely into the catalytic site of the ATP, resembling the binding of the adenosine moiety of the cofactor (FIG. 3 A,B,C). Indeed each compound is involved in H-bonding interactions with residues Lys181, Ala232, Thr292 and Thr162 (FIG. 3D) similarly to what observed in the crystal structure of Akt1 in complex with AMP-PNP. In FIG. 3, A), B) and C) Docked structures of compounds 1, 2 and 3 into the ATP binding site of Akt1. The 2D structures of 50000 compounds were converted to 3D structures using CONCORD (25) or CORINA (26). Two docking programs were used to screen compounds against Akt1 kinase. FlexX program applied Drugscore to determine docked conformers. GOLD package docked ligands using Goldscore fitness function. Consensus scoring was obtained by using CSCORE (Sybyl). D) Hydrogen-bonds between compound 1 and residues present in the Akt1 catalytic pocket.

Accordingly, measurement of inhibitory properties of additional 13 analogues of compound 1 revealed that only compounds 4 and 5, both capable of forming H-bonds with the above mentioned residues, showed appreciable inhibition in the micromolar range (Table 2).

Therefore the ability of a given compound to be involved in H-bond interaction appears to be essential in all the inhibitor-Akt 1 complexes as previously reported for other protein kinases (23) and in other docking studies (27). In fact, when the ability of forming H-bonds is taken into account for the selection of candidate inhibitors, only 30 compounds would be selected. As described in FIG. 1B, our selected 30 compounds contain all three hits thus yielding a hit rate of 10%.

Despite the availability of many reliable in silico approaches and robust in vitro commercially available assays, discovering Akt inhibitors still remains a challenging task. Even though several attempts have been performed in this field, there are presently no marketable inhibitors against Akt1 besides H-89. In fact, a very recent paper reports on a study, which based on high-throughput screening, led to the characterization of only two Akt1 low micromolar inhibitors out of 270,000 tested compounds (23).

Moreover, during our ongoing efforts to identify molecules capable of blocking Akt-kinases activity, we also tested a library of 2000 natural products (Microsource) at a concentration up to 30 μM against Akt1, but no compound emerged as an effective low micromolar inhibitor (data not shown).

In conclusion, in the present Example we describe two different structure-based strategies we adopted in order to discover compounds inhibiting Akt1. When we applied the strategy described in FIG. 1A, simply relying on results provided by the scoring functions, very disappointingly the hit rate appeared to be only slightly superior to the one expected from a random approach (0.01-0.5%) (24). However, when our docking methodology outcome was analyzed by taking into account a consensus between scoring functions and H-bonding patterns similar to those observed in the crystal structure of Akt1 in complex with AMP-PNP, a remarkable 10% hit rate was finally achieved (FIG. 1B).

We believe that the two low micromolar inhibitors described here may represent a starting point for finding potent and selective molecules capable of preventing Akt1 activity in human tumoral cells.

### Example 2

### Identification of BI-69A11 as AKT inhibitor

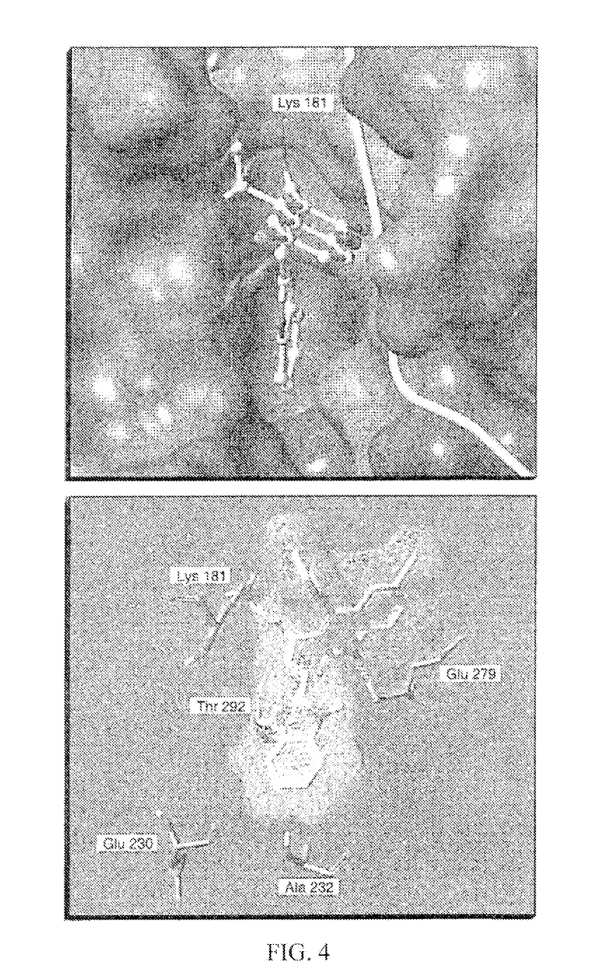

We recently reported on the direct evaluation of a number of in silico approaches to identify AKT inhibitors (Forino et al., 2005) We achieved experimental validation of selected compounds using both a fluorescence-based enzymatic assay and a substrate phosphorylation assay involving the protein GSK-3 (Forino et al., 2005). Briefly, the virtual docking approach consists of selecting the top 4,000 out of 50,000 docked compounds, using a variety of computational docking approaches, including a consensus score among two different scoring functions (Forino et al., 2005). Of those, 100 compounds were selected based on ranking and favorable docking geometry. Finally, 2 compounds were selected for further evaluation based on their ability to inhibit AKT activity with IC50 values in the low micromolar range. Compound BI-69A11 (FIG. 4) inhibited AKT1 in a concentration range comparable to that of H-89, a commercially available AKT inhibitor, yielding IC50 values of 2.3 micro.M, through an ATP competitive inhibition (Forino et al., 2005). BI-69A11 did not affect the activity of other protein kinases including Abl1, p38.alpha, JNK and PI3K, even at high concentrations of 100 micro.M.

Based on the docked geometry, and in agreement with our experimental data, without being bound it is believed that BI-69A11 fits in the catalytic site of the ATP, resembling the binding of the adenosine moiety of the cofactor (FIG. 4). The predicted binding mode of BI-69A11 in the ATP site of PKB/AKT (pdb: 106K) (Yang et al., 2002) suggests that it forms three hydrogen bonds with residues Lys181, Thr292 and Glu279 (FIG. 1). These would account for its inhibitory properties against AKT and for the benzimidazole ring occupying an adjacent hydrophobic region. These favorable inhibitory properties of BI-69A11 promoted further synthesis and cell-based evaluations of BI-69A11.

### Example 3

### Characterization of BI-69A11 in Melanoma Cells

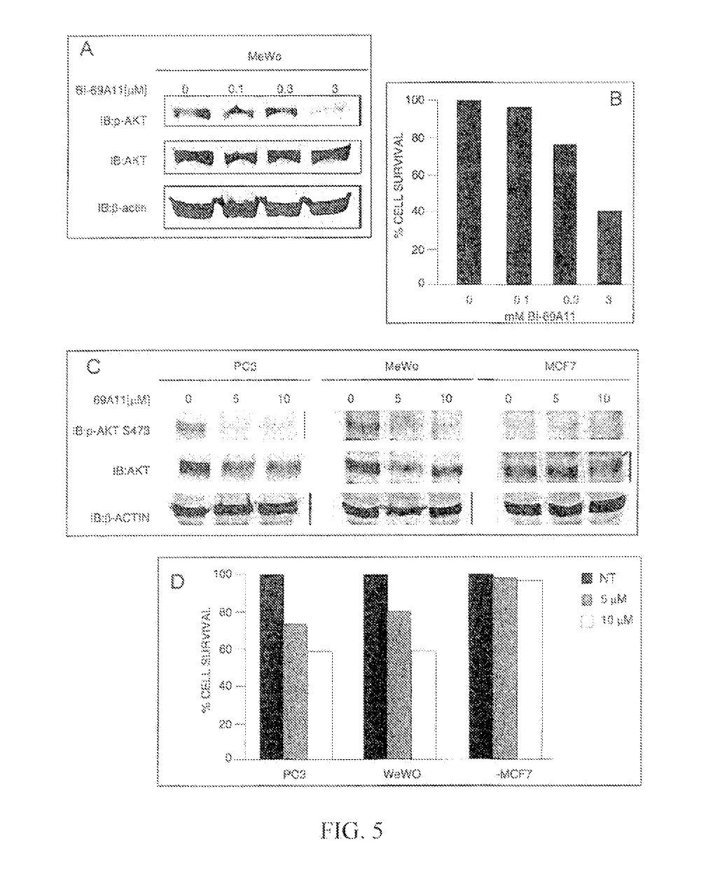

To evaluate the effectiveness of BI-69A11 on melanoma cells we assessed the effect of different concentrations on AKT phosphorylation in MeWO cells. While low doses (<0.3 micro.M) did not affect AKT phosphorylation, a dose of 3 micro.M BI-69A11 caused partial inhibition of AKT phosphorylation on S473, which serves as a marker for AKT activity (FIG. 5A). Analysis of cell death revealed that about 60% of the melanoma cells were dead within 24 hours after treatment with the 3 micro.M dose of BI-69A11 (FIG. 5B). These data provide initial support for the effectiveness of this inhibitor on AKT phosphorylation and melanoma cell death.

To substantiate these initial findings we have set to compare the effect of the BI-69A11 on AKT phosphorylation and cell death among melanoma, prostate and breast tumor cell lines. Since the concentration of 3 micro.M caused partial inhibition of AKT phosphorylation, we have now compared the effect of two higher concentrations of BI-69A11, 5 and 10 micro.M. Compared with MeWo melanoma cells, PC3 prostate tumor cells were equally affected upon treatment with BI-69A11. In both cases, the basal level of AKT phosphorylation was effectively inhibited by the 5 micro.M, as well as the 10 micro.M dose (FIG. 5C). Notably, the decrease in AKT phosphorylation at these doses of the BI-69A11 coincided with reduced levels of AKT protein (FIG. 5B). In contrast, AKT protein levels were not affected in MCF7 cells, which do not express a constitutively active AKT (Lu et al., 2006; FIG. 5B). These initial data suggest that AKT phosphorylation may be required for BI-69A11 to affect AKT, which causes a decrease in AKT protein levels, and which is also reflected at the level of its phosphorylation. Consistent with the effect of BI-69A11 on AKT phosphorylation was its effect on cell death. Within 4 hours of treatment with 5 micro.M of BI-69A11 about 25% of both PC3 and MeWo underwent cell death (FIG. 5D). Strikingly, such treatment did not affect viability of the MCF7 cells (FIG. 5D). These data suggest that BI-69A11 causes effective death of tumor cells that express an active form of AKT. Consistent with these observations, melanocytes that are grown in culture in the presence of growth factors express active AKT; this is no longer seen if the cultures are deprived of these factors for 12-24 hours. Growth factor-maintained melanocytes treated with BI-69A11 caused efficient inhibition of AKT phosphorylation, which coincided with cell death; whereas treatment of melanocytes deprived of growth factors (for 24 h) no longer elicit toxic effect on these melanocytes (data not shown).

### Example 4

### BI-69A11 Inhibits AKT Activity, AKT Protein Levels and AKT Association with HSP90

To further characterize the inhibition of AKT by BI-69A11 we used a set of melanoma cells in which PTEN is either inactive (UACC 903 cells) or active due to reconstitution of chromosome 10 with wt PTEN (clone 29-1; Robertson et al., 1998).

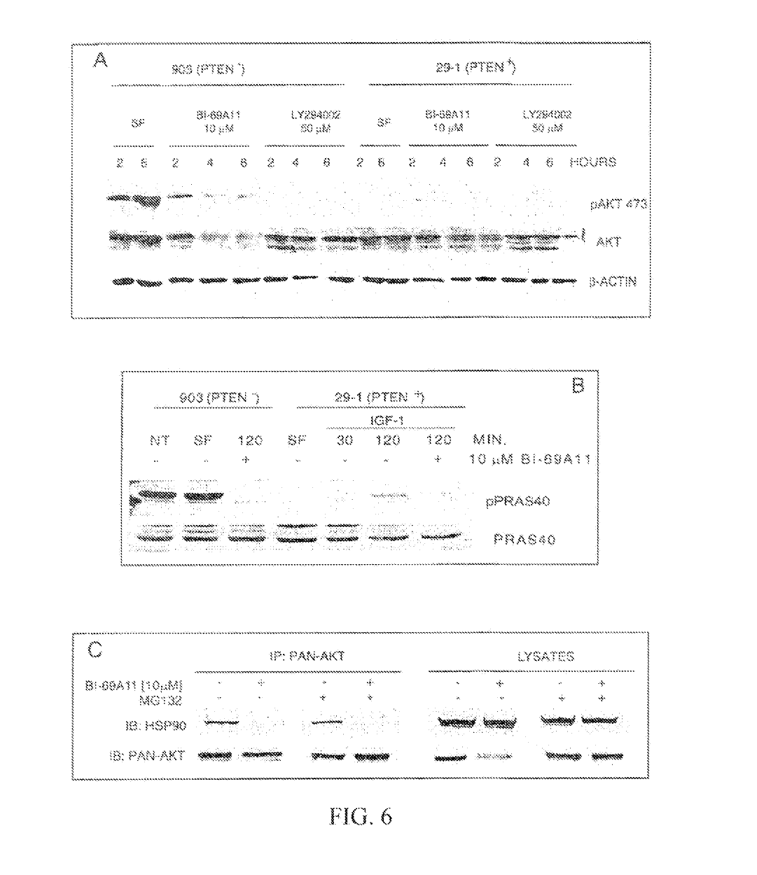

The addition of BI-69A11 to UACC 903 cells caused a dose-dependent decrease in the levels of pAKT Ser473 (FIG. 6A). Thus, we set to test the possibility that BI-69A11's ability to inhibit AKT activity may stem from inhibition of AKT protein expression (see data below). We thus assessed whether inhibition of AKT Ser473 phosphorylation coincides with its activity, namely the phosphorylation of known AKT substrates. As shown, phosphorylation of PRAS40 was markedly inhibited in these cells (FIG. 6B). Of note, AKT3, the primarily active isoform of AKT in melanoma cells, effectively phosphorylates PRAS40 (Madhunapantula et al., 2007).

We next tested the effect of BI-69A11 in 29-1 cells which are derivative of UAC903 that were reconstituted with chromosome 10 carrying the wt PTEN. Thus, 29-1 cells no longer express a constitutively active form of AKT (Robertson et al., 1998). To activate AKT, 29-1 cells were treated with IGF-1 (FIG. 6B). While resulting in the activation of AKT and concomitant phosphorylation of PRAS40 (although to a lesser degree when compared with the UACC903 cells), the addition of BI-69A11 effectively inhibited the phosphorylation of PRAS40 (FIG. 6B). These results suggest BI-69A11 may serve as a potent inhibitor of AKT activity.

Given the effect of BI-69A11 on the level of AKT protein, we assessed the possible mechanisms underlying such an effect. First, we assessed whether BI-69A11 affects the level of AKT transcripts. RNA prepared from the melanoma cells, with and without treatment with BI-69A11, was subjected to QPCR analysis. Such analysis did not identify differences in AKT transcripts (data not shown) suggesting that the inhibitor does not affect the mRNA of AKT.

Among cellular proteins implicated in the regulation of AKT levels is the cellular chaperone HSP90. Inhibition of HSP90 chaperone function, as commonly achieved by the geldanamycin antibiotic, interferes with the conformational maturation and refolding of its associated proteins, thereby promoting their degradation (Munster et al., 2002; Neckers and Neckers, 2003). AKT was shown to be affected either directly or indirectly by inhibition of HSP90 (Basso et al., 2002; Xu et al., 2003; Fujita et al., 2002; Theodoraki et al., 2007). Thus we assessed whether association of AKT with HSP90 could be affected by BI-69A11. Immunoprecipitation of AKT from melanoma cells identified HSP90 in the immunoprecipitated material. Such association was however abolished following treatment with the BI-69A11 inhibitor (FIG. 6C). Since BI-69A11 causes reduced level of AKT protein (FIG. 2C, 3A, 3C lysate panel) we wanted to monitor such association under conditions in which AKT levels are comparable. To this end, we also performed the experiment in the presence of the proteasome inhibitor MG132. Although MG132 restored the level of AKT protein expression, it did not restore HSP90-association with AKT (FIG. 6C). These data suggest that the decrease in AKT levels seen following treatment with BI-69A11 can be attributed to disrupted association with the HSP90 chaperone. To determine whether BI-69A11 may also affect other HSP90 client proteins, we have monitored changes in the levels of p53 and c-Jun, two short lived proteins that were shown to be among HSP90 client proteins whose stability is affected by geldanamycin. BI-69A11 did not affect c-Jun protein levels, and had limited effect on the level of p53 (data not shown). These data imply that BI-69A11 primarily affects AKT, which interferes with its ability to associate with HSP90.

### Example 5

### BI-69A11 Elicits More Effective Cell Death in Melanoma Cells Expressing Active AKT

Since AKT activity is important for tumor cell survival, its inhibition is expected to result in cell death. We thus monitored cell death in melanoma cells that were treated with BI-69A11. With concentrations as low as 1 micro.M, BI-69A11 caused effective cell death within 24 hours following treatment. Interestingly, the effect of the inhibitor was more pronounced on PTEN mutant melanoma cells, compared with the 29-1 cells into which the wt PTEN was reconstituted (FIG. 7A). Similarly, when compared to the earlier time point, BI-69A11 exhibited a stronger effect on the PTEN mutant melanomas, albeit a higher dose of the inhibitor was required to achieve cell death within the 6-hour time point (FIG. 7A). Consistent with these findings, PARP cleavage was more pronounced in the UAC903 cells, compared with the 29-1 line (FIG. 7B). These data point to a more efficient effect of BI-69A11 on melanoma cells that harbor higher AKT activity.

### Example 6

### BI-69A11 Inhibits Melanoma Xenografts

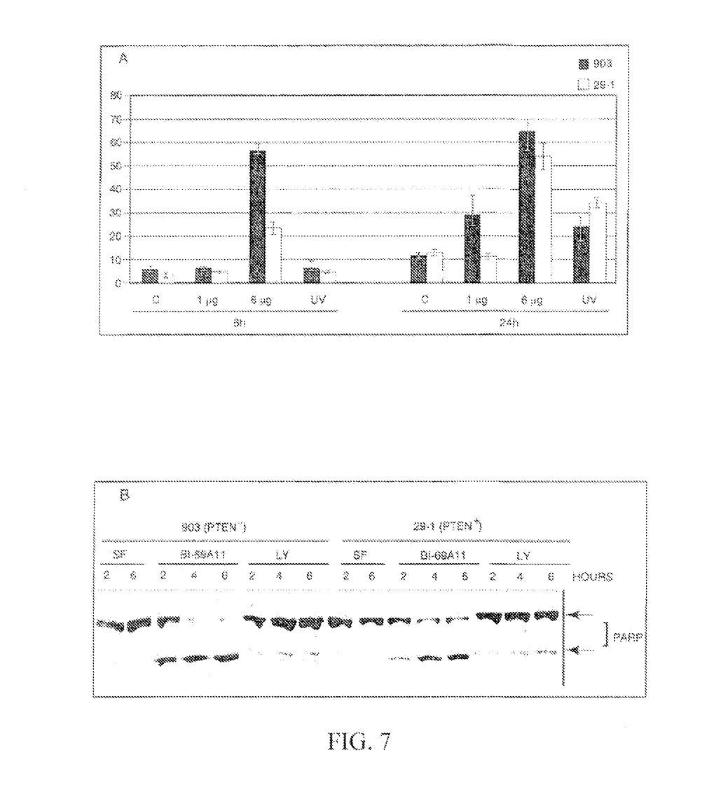

In light of the efficient cell death elicited by BI-69A11, we assessed its activity on tumor growth in mouse xenografts. To this end, 903 human melanoma cells, which harbor a PTEN mutation, were injected subcutaneously into nude mice and tumors were allowed to form over a period of 10 days. When the tumors reached an approximate size of 1 mm.sup.3, mice were injected intra-peritoneally twice per week with BI-69A11 (0.5-2.0 mg/kg), or with a control (DMSO; 0.1%, used to dissolve the inhibitor). The selection of this dose-range for BI-69A11 was based on initial MTD assays, which revealed toxicity at the 5 mg/kg dose (data not shown). Strikingly, BI-69A11 effectively inhibits further growth of the melanoma tumors. Furthermore, treatment with the inhibitor caused efficient regression in these tumors (FIG. 8A). Interestingly, lower concentrations of BI-69A11 were as efficient, and perhaps slightly more so, at inhibition of melanoma growth, compared with the higher ones (FIG. 8A). Of note, removal of the inhibitor 3 days prior to termination of the experiment resulted in an initial increase in tumor size in mice that were treated with the low concentration of the inhibitor, but not in those that received the higher doses (compare 22 and 24 day bars in FIG. 8A). Using Tunel staining, analysis of the tumors from each of the treatment groups for apoptosis revealed higher degrees of apoptosis in the treatment groups compared with the control tumors. Strikingly, the tumors that were obtained from the lower-dose treatment exhibited higher levels of apoptosis (FIG. 6B, 6C). These data suggest that BI-69A11-induced apoptosis is, in part, a mechanism by which it causes regression of the melanoma tumors tested here. The data also suggest that BI-69A11 elicits effective inhibition of melanoma tumor growth in vivo.

The present invention elucidates the activities of BI-69A11, a competitive inhibitor for AKT that was identified by using a virtual docking approach based on consensus scoring (Forino et al., 2005). Expected properties for this inhibitor, based on initial analysis, predicted competitive inhibition against AKT. Analysis of the inhibitor for its biological activities was made in melanoma cells in which AKT is commonly hyperactive. Using the 903 melanoma cell system we were able to compare the activities of BI-69A11 between the original cells where PTEN is deleted to those in which PTEN was reconstituted (clone 29-1). Clearly, while BI-69A11 effectively blocked AKT phosphorylation in the 903 cells, there was limited to no effect on the 29-1 cells. Of importance, the inhibitor was able to block IGF-induced AKT phosphorylation of PRAS40, a known downstream target of AKT, in the 29-1 cells, suggesting that it is capable of affecting physiological stimuli elicited in PTEN wt cells. BI-69A11 was as efficient in inhibition of AKT phosphorylation in other melanoma cell lines, including MeWo, as it was in the prostate tumor PC3 cells.

Importantly, BI-69A11's effect on AKT phosphorylation appears to be coupled with its effect on AKT protein levels. Our initial analysis reveals that AKT association with HSP90 is inhibited in melanoma cells that were treated with BI-69A11. As inhibition of the HSP90 complex with client proteins has been shown to affect their stability, this finding is likely to explain the nature of reduced AKT protein in BI-69A11 treated cells. Consistent with these observations, recent evidence indicates that mTORC2, a complex consisting of mTOR, SIN1, mLST8 and rictor, shown to phosphorylate AKT at Ser473, also causes stabilization of AKT through interaction with HSP90, thus facilitating its protection through ubiquitin mediated degradation (Facchinetti et al., 2008). Without being bound we propose that dephosphorylation of AKT at this residue disables AKT-HSP90 association, rendering AKT unstable. The latter is consistent with initial analyses of other HSP90 client proteins whose stability is not affected by BI-69A11 compared with AKT.

Significantly, inhibition by BI-69A11 resulted in apoptosis of melanoma cells, consistent with previous reports of the effect of inhibiting AKT in such cells. Similarly, BI-69A11 caused efficient cell death in PC3 prostate tumor cells, which express active AKT. Of note, the degree of inhibition was greater in the tumor cells that harbor an active form of AKT, in agreement with the primary effect of this inhibitor on AKT activity. Consistent with these observations, the inhibitor did not affect the viability of MCF7 breast cancer cells, which do not express a constitutively active form of AKT, nor did it affect melanocytes that were deprived of growth factors, which would otherwise induce AKT activity. These findings substantiate the effectiveness of BI-69A11 on active AKT expressing cells. Significantly, BI-69A11 efficiently inhibited melanoma tumor growth and metastasis in a xenograft model. Overall, the present study provides initial characterization of an AKT inhibitor that affects select AKT signaling pathways. The effective cell death and inhibition of tumor growth in xenograft models justifies further studies of BI-69A11 for pre-clinical and clinical evaluations.

### Example 7

### BI-69A11 Inhibits the NFkB Pathway

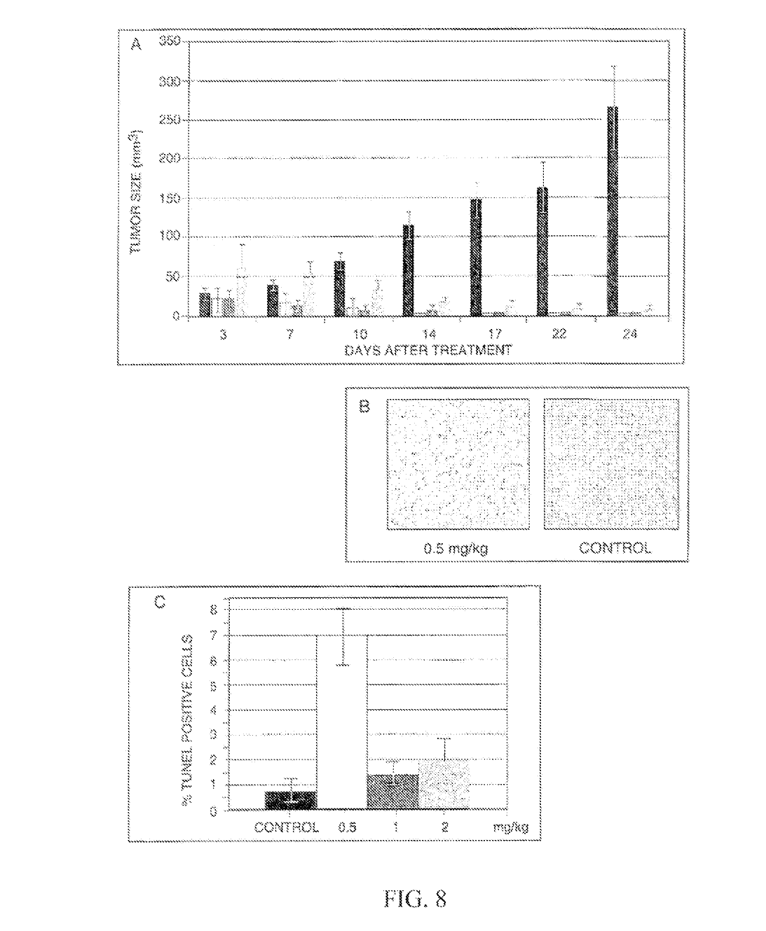

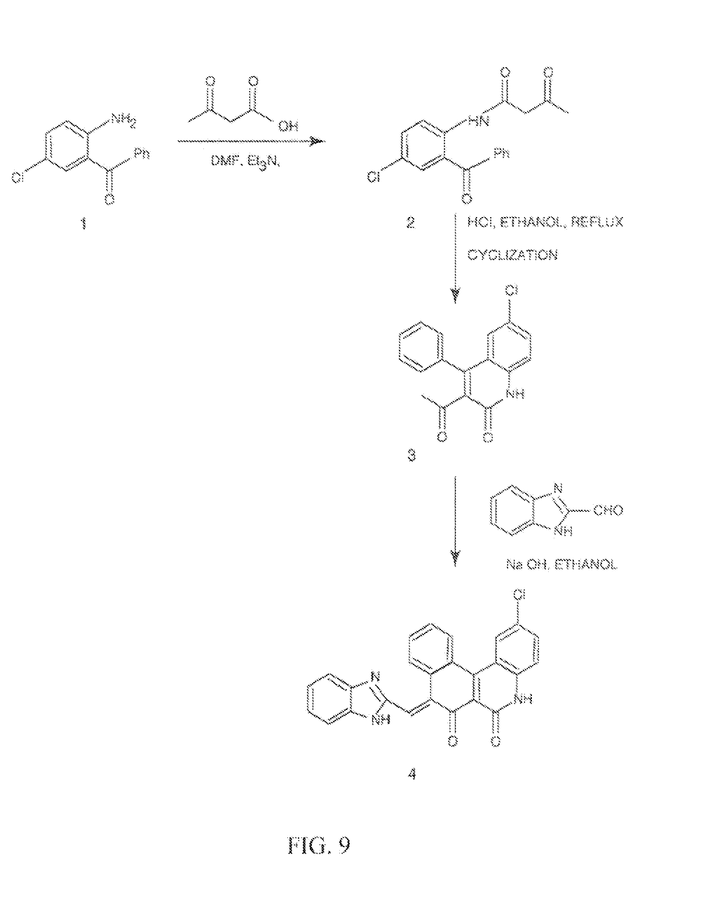

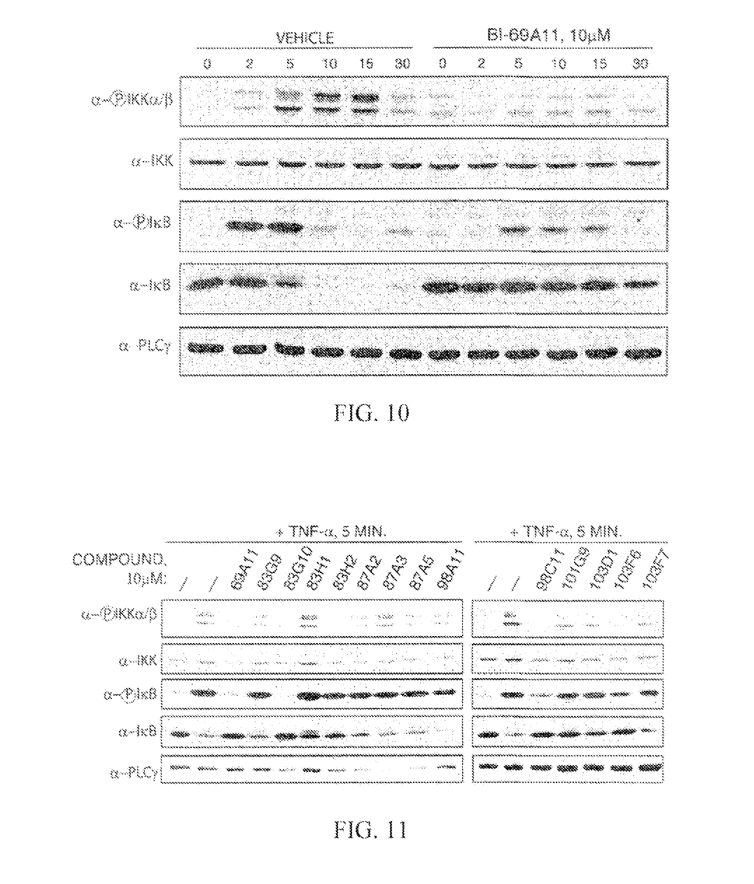

We first tested whether BI-69A11 inhibited the activation of the canonical NF-kB pathway (FIG. 10) in intact cells. Stimulation of vehicle-treated UACC903 melanoma cells with an NF-kB activator, tumor necrosis factor-alpha (TNF-a) resulted in the phosphorylation and activation of IKK and led to the phosphorylation and subsequent degradation of IkB within 2-5 minutes. In contrast, in cells treated with BI-69A11, both IKK and IkB phosphorylation were inhibited in response to TNF-a stimulation. Furthermore, the degradation of IkB protein was inhibited. These data suggest that BI-69A11 inhibits the NF-kB pathway.

### Example 8

### Selected BI-69A11 Analogs Inhibit the NFkB Pathway

We next tested the ability of selected BI-69A11 analogs to inhibit the NF-kB pathway (FIG. 11). In this experiment, UACC903 cells were treated with vehicle, BI-69A11 or analog, stimulated with TNF-a, and harvested at 5 minutes post-stimulation. As in the previous experiment, BI-69A11 inhibited IKK and IkB phosphorylation. The BI-69A11 analog, BI-83G10, also showed inhibition equal to that of BI-69A11, while BI-98C11 also showed inhibition, albeit to a lesser extent. The other analogs tested (BI-83H1, -83H2, -87A2, -87A3, -87A5, -98A11, -101G9, -103D1, -103F6, -103F7) did not inhibit NF-kB signaling.

### Example 9

### Decreased Cell Viability in the Presence of BI-69A11 or the Analogs BI-83G10 and BI-98C11

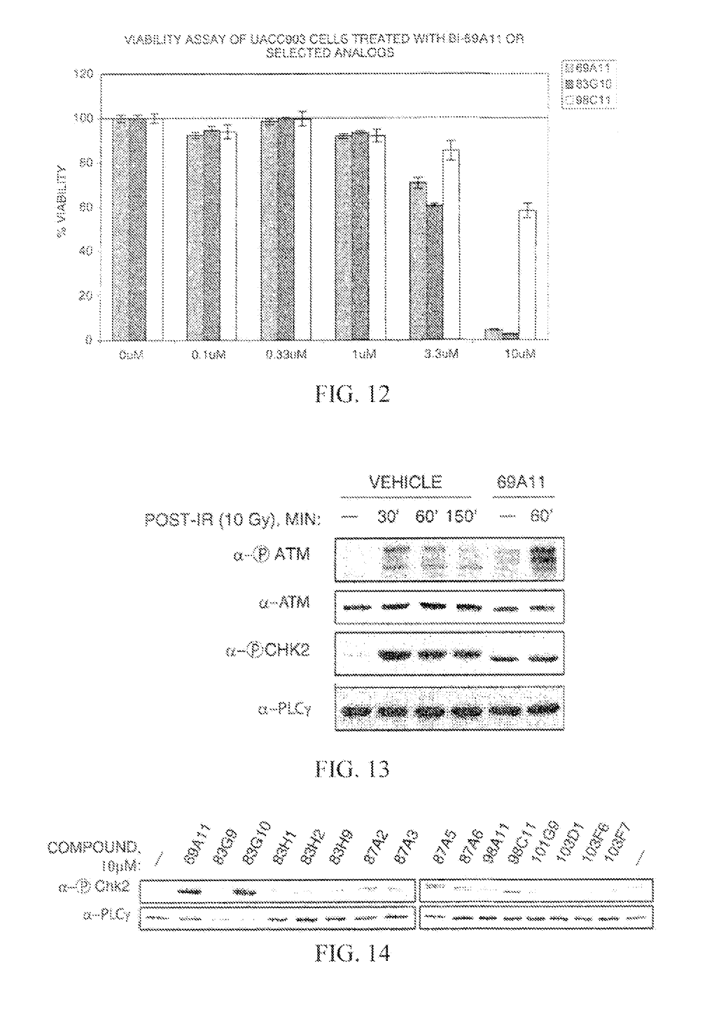

As BI-69A11 decreases melanoma cell viability, we next addressed whether the inhibition of NF-kB by BI-69A11 or the analogs BI-83G10 and BI-98C11, showed any correlation with cell viability (FIG. 12). In accordance with previous results, BI-69A11 decreased the viability of UACC903 cells, while BI-83G10 showed a similar effect. Interestingly, BI-98C11 showed a decreased effect on cell viability compared to BI-69A11 or -83G10. Taken together with the biochemical data, these results suggest that cell viability in the presence of BI-69A11 correlates with the degree of NF-kB inhibition, and implicates this pathway as an additional target of BI-69A11.

### Example 10

### BI-69A11 Activates the DNA Damage Response

As the Invitrogen SelectScreen identified CHK2 as a possible target of BI-69A11, we tested whether DNA damage-induced CHK2 activation was inhibited by BI-69A11 in intact cells. To accomplish this, we g-irradiated vehicle- or BI69A11-treated cells and examined ATM and CHK2 phosphorylation, a well-characterized surrogate of activation (FIG. 13). In vehicle-treated cells, g-irradiation led to the robust phosphorylation of ATM and CHK2. However, BI-69A11 did not inhibit ATM or CHK2 phosphorylation following g-irradiation, suggesting that it does not inhibit CHK2. Instead, treatment of cells with BI-69A11 in the absence of radiation surprisingly leads to ATM and CHK2 phosphorylation, suggesting that BI-69A11 activates a DNA damage response.

### Example 11

### Assessment of BI-69A11 Analogs on Chk2 Phosphorylation

We next examined the panel of BI-69A11 analogs for their ability to stimulate CHK2 phosphorylation in UACC903 cells (FIG. 14). Similar to our results in the NF-kB assay, both BI-69A11 and BI-83G10 induced the activation of CHK2 to a similar extent, while BI-98C11 induced CHK2 activation to a lesser extent. These data suggest that BI-69A11 can also elicit the DNA damage response mediated by the ATM-CHK2 axis.

### Example 12

### Administration of BI-69A11 NRAS/Ink4a Mice

The NRAS/Ink4a mice harbor a Q61K NRAS activating mutation, as is seen in 15% of human melanomas, on an Ink4a-deficient background, which mimics the mutations often seen in this locus (p16) in human melanoma.

The NRAS/Ink4a mouse develops melanoma with penetrance of about 25-50% which is often accompanied by metastasis to the lymph nodes. Melanoma develops in this model as early as 15 weeks after induction of NRAS expression, with most melanoma appearing between 20 and 40 weeks post-induction Recently, this model was used to demonstrate the important role of ATF2 as oncogene in melanoma (Shah et al., PLoS Genet. 6(12): e1001258. doi:10.1371/journal.pgen.1001258).

To examine the effect of BI-69A11 treatment on in vivo melanoma development, BI-69A11 was administered to a cohort of 30 mice twice per week (IP, 0.5 mg/kg), starting at week 28 after NRAS expression. A control group of 30 mice received the control vehicle formulation. The data for up to 40 weeks are shown in FIG. 16A. In this model, by 28 weeks many mice already developed melanoma. Those were not included in our study which was restricted to animals that have not yet developed melanoma. Nevertheless, in a clinical setting, preventive treatment could be administered prior to tumor development, at a stage where early stages of development had been initiated.

NRAS expression was induced in NRAS/INK4a mice (time 0) and the mice were maintained for 40 weeks. Starting at 28 weeks, animals received BI-69A11 at 0.5 mg/kg by IP injection twice weekly. The graph shows the total number of melanomas, measured weekly. Tumors appeared in this model as early as week 12, indicating that at the time we began this assessment, tumors were well into their development phase.

A total of 19 tumors developed in mice receiving BI-69A11, compared with 30 tumors in the control group over the same time period. Of the combined total of 49 tumors detected, 24 were internal and are likely to be fibrosarcomas, which in this model constitute about 50% of the tumors. The remaining 25 tumors were confirmed to be melanomas by IHC of the melanocyte markers DCT and S100. Of these 25, 17 (68%) developed in the control formulation-treated animals, whereas only 8 (32%) in the BI-69A11-treated group. See FIG. 16B. Collectively, these data demonstrate that treatment with BI-69A11 halted the development of melanomas in the NRAS/Ink4a model.

NRAS expression was induced in NRAS/INK4a mice and 28 weeks later treatment with BI-69A11 was initiated. Internal tumors developed in this model were fibrosarcomas, whereas the skin tumors were melanomas (confirmed by immunostaining with S100 and DCT). The control group developed 17 melanomas, compared with 8 melanomas in the BI-69A11 group (˜68% inhibition). Less inhibition was seen in the fibrosarcoma group (˜50%).

An additional experimental group included animals from the same melanoma model, albeit, those were treated earlier (after 10 weeks rather than 28). Our data indicated that the prevention seen when BI-69A11 was administered after 28 weeks was sustained, and that administration of the compound after 10 weeks was as effective as administration of the compound after 28 weeks.

Blood work performed on the animals that received BI69A11 for over 3 months indicated lack of toxicity; all evidence suggested that the drug is well tolerated. Analysis of cytokines in the blood of treated vs. control treated mice pointed to induction of immune response by BI-69A11, which is reflected in induction of TNFalpha seen in the treated but not the control compound administered group.

Collectively, the data point to the possible use of BI69A11 for prevention of melanoma bearing N-Ras mutation. Notably, this is among the subgroups of melanoma to which there are no known effective therapies. Hence, the possible advance of 69A11 to pre-IND and clinical trials is of major interest and importance.

For western blotting techniques, cells were lysed in RIPA buffer containing 50 mM TrisHCl pH7.4, 150 mM NaCl, 2 mM EDTA, 1% NP-40, 0.1% SDS. The protein concentration was estimated using the Coomassie Protein assay kit (Thermo Scientific, Rockford, Ill., USA). Equal concentrations of cell lysate (30-50 ug) were resolved on a 10% polyacrylamide gel and transferred onto Nitrocellulose membrane (GE Amersham). Membranes were blocked for 1-2 hours in either 3% BSA or 5% nonfat dried milk in Tris-buffered saline containing 0.05% Tween 20. Primary antibody incubations were performed overnight at 4° C. with shaking. The blots were washed followed by incubation with a secondary antibody incubation for 1 hour at room temperature. After washing, the blots were scanned on the Odyssey Licor scanner at the appropriate wavelength and the image was captured using the Odyssey software.

For antibody generation, pAKT473 was obtained from Cell Signaling Technologies (Beverly, Mass., USA), total AKT, pPRAS40 and total PRAS40 from Biosource, PARP (Cell Signaling Technologies (Beverly, Mass., USA), c-Jun and p53 (Santa Cruz Biotechnology, Santa Cruz, Calif., USA), HSP-90 (Abeam, Cambridge, Mass., USA), and actin (Sigma, St. Louis, Mo.). Secondary antibodies were goat anti-rabbit coupled to Alexafluor 580 and goat anti-mouse coupled to Alexafluor 800.

For the proliferation assays, cells were treated as described above. However, instead of scraping cells into the serum-free medium, floating cells were collected in a 15 mL centrifuge tube whereas adherent cells trypsinized gently. The total cell population was pooled and counted on a hemocytometer after staining with Trypan Blue. The percentage of dead cells was determined by calculating the number of dead cells/total cells X 100. This experiment was performed two times each in triplicate.

For the tumor studies, UACC 903 cells (1×106) were injected subcutaneously and tumor size was monitored using calipers twice per week. Administration of BI-69A11 was performed twice weekly, via IP injection, when tumors reached an approximate size of 1 mm sup.3. Mice were injected with different concentrations (0.5 mg/kg, 1.0 mg/kg, 2.0 mg/kg) of BI-69A11, or with control DMSO (0.1%) used to dissolve the inhibitor. All injections were put into a mixture of ethanol and cremophor (1:1) and suspended in saline (ethanol—cremophor 10% final concentration) for a total of 300 micro.1 per injection. Intra-peritoneal injections were performed twice weekly, for a period of 3 weeks. At the end of the study, tumors were harvested, weighed and measured.

For the tunel assay and quantitative analysis, the detection of nuclei with fragmented DNA by terminal deoxyribonucleotidyl transferase-mediated dUTP nick end labeling (TUNEL) was accomplished using the ApopTag Peroxidase In situ Apoptosis Detection Kit (Chemicon, Temecula, Calif., US) according to the instructions of the manufacturer. Meyers Hematoxylin was used as a counterstain. Slides were scanned at 40× magnification (resolution of 0.25 micro.m/pixel [100,000 pix/in.]) using the Aperio ScanScope® CS system (Aperio Technologies, CA, US). Spectrum Analytics package with nuclear algorithm and analysis software Image Scope (Aperio) were applied to quantify tunnel positive cells present in the entire cross sections of tumor specimens.

For the computer modeling, the predicted binding mode of BI-69A11 in the ATP pocket of AKT (pdb:1O6K) was generated using the docking software GOLD (GOLD, version 3.2. The Cambridge Crystallographic Data Centre, Cambridge, UK).

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

### REFERENCES

Klingmuller, U. The role of tyrosine phosphorylation in proliferation and maturation of erythroid progenitor cells—signals emanating from the erythropoietin receptor. Eur. J. Biochem. 1997, 249, 637-647.
Lev, D. C.; Kim, L. S.; Melnikova, V.; Ruiz, M.; Ananthaswamy, H. N.; Price, J. E. Dual blockade of EGFR and ERK1/2 phosphorylation potentiates growth inhibition of breast cancer cells. Brit. J. of Cancer 2004, 91, 795-802.
Dickson, L. M.; Rhodes, C. J. Pancreatic beta-cell growth and survival in the onset of type 2 diabetes: a role for protein kinase B in the Akt? Am. J. Physiol. Endocrinol. Metab. 2004, 287, 192-198.
Okano, J.; Gaslightwala, I.; Birnbaum, M. J.; Rustgi, A. K.; Nakagawa, H. Akt/protein kinase B isoforms are differentially regulated by epidermal growth factor stimulation. J. Biol. Chem. 2000, 275, 30934-33942.
Chijiwa, T.; Mishima, A.; Hagiwara, M.; Sano, M.; Hayashi, K.; Inoue, T.; Naito, K.; Toshioka, T.; Hidaka H. Inhibition of forskolin-induced neurite outgrowth and protein phosphorylation by a newly synthesized selective inhibitor of cyclic AMP-dependent protein kinase, N-[2-(p-bromocinnamylamino)ethyl]-5-isoquinolinesulfonamide (H-89), of PC12D pheochromocytoma cells. J. Biol. Chem. 1990, 265, 5267-5272.
Stratford, S., Hoehn, K. L., Liu, F., and Summers, S. A. Regulation of insulin action by ceramide: dual mechanisms linking ceramide accumulation to the inhibition of Akt/protein kinase B. J. Biol. Chem. 2004, 279, 36608-36615.
Baxter, C. A.; Murray, C. W.; Waszkowycz, B.; Li, J.; Sykes, R. A.; Bone, R. G. A.; Perkins, T. D. J.; Wylie, W. New approach to molecular docking and its application to virtual screening of chemical databases. J. Chem. Inf. Comput. Sci. 2000, 40, 254-262.
Carr, R.; Jhoti, H. Structure-based screening of low-affinity compounds. Drug Discov. Today 2002, 7, 522-527.
Perola, E.; Xu, K.; Kollmeyer, T. M.; Kaufmann, S. H.; Prendergast, F. G.; Pang, Y. P. Successful Virtual Screening of a Chemical Database for Farnesyltransferase Inhibitor Leads. J. Med. Chem. 2000, 43, 401-408.
Yang, J.; Cron, P.; Good, V. M.; Thompson, V.; Hemmings, B. A.; Barford, D. Crystal Structure of an Activated Akt/Protein Kinase B Ternary Complex with Gsk-3 Peptide and AMP-PNP. Nat. Struct. Biol. 2002, 9, 940.
SYBYL. 6.9. 1699 South Hanley Road, St. Louis, Mo., 63144, USA: Tripos Inc. Rarey, M.; Kramer, B.; Lengauer, T.; Klebe, G. A fast flexible docking method using an incremental construction algorithm. J. Mol. Biol. 1996, 261, 470-489.
FlexX 1.13.5. Sankt Augustin, Germany BioSolveIT GmbH.
Clark, R. D.; Strizhev, A.; Leonard, J. M.; Blake, J. F.; Matthew, J. B. Consensus scoring for ligand/protein interactions. J. Mol. Graph. Model. 2002, 20, 281-295.
Gohlke, H.; Hendlich, M.; Klebe, G. Knowledge-based Scoring Function to Predict Protein-Ligand Interactions. J. Mol. Biol. 2000, 295, 337-356.
Jones, G.; Willett, P.; Glen, R. C.; Leach, A. R.; Taylor, R. Development and validation of a genetic algorithm for flexible docking. J. Mol. Biol. 1997, 267, 727-748.
Eldridge, M. D.; Murray, C. W.; Auton, T. R.; Paolini, G. V.; Mee, R. P. Empirical scoring functions: 1. The development of a fast empirical scoring function to estimate the binding affinity of ligands in receptor complexes. J. Comput-Aided Mol. Des. 1997, 11, 425-445.
GOLD. 2.1. 12 Union Road, Cambridge, CB2 1 EZ, UK: The Cambridge Crystallographic Data Centre.
Rodems, S. M.; Hamman, B. D.; Lin, C.; Zhao, J.; Shah, S.; Heidary, D.; Makings, L.; Stack, J. H.; Pollok, B. A. A FRET-Based Assay Platform for Ultra-High Density Drug Screening of Protein Kinases and Phosphatases. ASSAY and Drug Development Technologies 2001, 1.
Reuveni, H.; Livnah, N.; Geiger, T.; Klein, S.; Ohne, O.; Cohen, I.; Benhar, M.; Gellerman, G.; Levitzki, A. Toward a PKB inhibitor: modification of a selective PKA inhibitor by rational design. Biochemistry 2002, 41, 10304-10314.
McGovern et al., J. Med. Chem. 45, 1712-22 (2002).
Daub, H.; Specht, K; Ullrich, A. Strategies to overcome resistance to targeted protein kinase inhibitors. Nature reviews, 2004, 3, 1001-1010.
Barnet, S. F.; Defeo-Jones, D.; Fu, S.; Hancock, P. J.; Haskell, K. M.; Jones, R. E.; Kahana, J. A.; Kral, A. M.; Leander, K.; Lee, L. L.; Malinowski, J.; McAvoy, E. M.; Nahas, D. D.; Robinson, R. G.; Huber, H. E. Identification and characterization of pleckstrin homology domain dependent and isozyme specific Akt inhibitors. Biochemical Journal Immediate Publication. Published on 29 Sep. 2004 as manuscript BJ20041140.
Doman, T. N.; et al. Molecular docking and high-throughput screening for novel inhibitors of protein tyrosine phosphatase-1B. J. Med. Chem. 2002, 45, 2213-2221.
Pearlman, R. S. Rapid Generation of High Quality Approximate 3-dimension Molecular Structures. Chem. Des. Auto. News, 1987, 2,1.
Sadowski, J.; Gasteiger, J.; Klebe, G. J. Comparison of Automatic Three-Dimensional Model Builders Using 639 X-ray Structures. Chem. Inf. Comput. Sci. 1994, 34, 1000-1008.
Examination of docked ligand orientation sampling methodology: mapping a pharmacophore for success. J. Mol. Graph. Model. 2003, 22(1), 31-40.
Basso, A. D., Solit, D. B., Chiosis, G., Gini, B., Tsichlis, P. and Rosen, N. (2002). AKT forms an intracellular complex with heat shock protein 90 (Hsp90) and Cdc37 and is destabilized by inhibitors of Hsp90 function. J. Biol. Chem. 277, 39858-66.
Carnero, A., Blanco-Aparicio, C., Renner, O., Link, W. and Leal, J. F. (2008). The PTEN/PI3K/AKT signalling pathway in cancer, therapeutic implications. Curr. Cancer Drug Targets. 8, 187-98.
Carracedo, A. & Pandolfi, P. P. (2008). The PTEN-PI3K pathway: of feedbacks and cross-talks. Oncogene. 27, 5527-41.
Cheung, M., Sharma, A., Madhunapantula, S. V. and Robertson, G. P. (2008). AKT3 and mutant V600E B-Raf cooperate to promote early melanoma development. Cancer Res. 68, 3429-39.
Cicenas, J. (2008). The potential role of AKT phosphorylation in human cancers. Int. J. Biol. Markers. 23, 1-9.
Dai, D. L., Martinka, M. and Li, G. (2005). Prognostic significance of activated AKT expression in melanoma: a clinicopathologic study of 292 cases. J. Clin. Oncol. 23, 1473-82.
De, S. K. and Gibbs, R. A. (2005). A mild and efficient one-step synthesis of quinolines. Tetrahedron Lett. 46, 1647-49.
Facchinetti, V., Ouyang, W., Wei, H., Soto, N., Lazorchak, A., Gould, C., Lowry, C., Newton, A. C., Mao, Y., Miao, R. Q., et al. (2008). The mammalian target of rapamycin complex 2 controls folding and stability of AKT and protein kinase C. EMBO. J. 27, 1932-43.
Forino, M., Jung, D., Easton, J. B., Houghton, P. J. and Pellecchia, M. (2005). Virtual docking approaches to protein kinase B inhibition. J. Med. Chem. 48, 2278-81.
Fried L. and Arbiser, J. L. (2008). The reactive oxygen-driven tumor: relevance to melanoma. Pigment Cell Melanoma Res. 21, 117-22.
Fujita, N., Sato, S., Ishida, A. and Tsuruo, T. (2002). Involvement of Hsp90 in signaling and stability of 3-phosphoinositide-dependent kinase-1. J. Biol. Chem. 277, 10346-53.
Garcia-Echeverria, C. and Sellers, W. R. (2008). Drug discovery approaches targeting the PI3K/Akt pathway in cancer. Oncogene. 27, 5511-26.
Goel, V. K., Lazar, A. J., Warneke, C. L., Redston, M. S, and Haluska, F. G. (2006). Examination of mutations in BRAF, NRAS, and PTEN in primary cutaneous melanoma. J. Invest. Dermatol. 126, 154-60.
Govindarajan, B., Sligh, J. E., Vincent, B. J., Li, M., Canter, J. A., Nickoloff, B. J., Rodenburg, R. J., Smeitink, J. A., Oberley, L., Zhang, Y., et al. (2007). Overexpression of AKT converts radial growth melanoma to vertical growth melanoma. J. Clin. Invest. 117, 719-29.
Haluska, F. G., Tsao, H., Wu, H., Haluska, F. S., Lazar, A. and Goel, V. (2006). Genetic alterations in signaling pathways in melanoma. Clin. Cancer Res. 12, 2301s-2307s.
Kudoh, et al., (2005). J. Biol. Chem., 280; 8156-8163.
Lindsley, C. W., Barnett, S. F., Layton, M. E. and Bilodeau, M. T. (2008). The PI3K/AKT pathway: recent progress in the development of ATP-competitive and allosteric AKT kinase inhibitors. Curr. Cancer Drug Targets. 8, 7-18.
Lu, D., Huang, J. and Basu, A. (2006). Protein kinase Cepsilon activates protein kinase B/Akt via DNA-PK to protect against tumor necrosis factor-alpha-induced cell death. J. Biol. Chem. 281, 22799-807.
Madhunapantula, S. V., Sharma, A. and Robertson, G. P. (2007). PRAS40 deregulates apoptosis in malignant melanoma. Cancer Res. 67, 3626-36.
Maniatis, (1999). Genes Dev., 13: 505.
May and Ghosh, seminars in Cancer Biology, 1997, 8: 63-73.
Munster, P. N., Marchion, D. C., Basso, A. D. and Rosen, N. (2002). Degradation of HER2 by ansamycins induces growth arrest and apoptosis in cells with HER2 overexpression via a HER3, phosphatidylinositol 3′-kinase-AKT-dependent pathway. Cancer Res. 62, 3132-7.
Neckers, L. and Neckers, K. (2002). Heat-shock protein 90 inhibitors as novel cancer chemotherapeutic agents. Expert Opin. Emerg. Drugs. 7, 277-88.
Robertson, G. P. (2005). Functional and therapeutic significance of AKT deregulation in malignant melanoma. Cancer Metastasis Rev. 24, 273-85.
Robertson, G. P., Furnari, F. B., Miele, M. E., Glendening, M. J., Welch, D. R., Fountain, J. W., Lugo, T. G., Huang, H. J. and Cavenee, W. K. (1998). In vitro loss of heterozygosity targets the PTEN/MMAC1 gene in melanoma. Proc. Natl. Acad. Sci. U.S.A. 95, 9418-23.
Sheng, S., Qiao, M. and Pardee, A. B. (2008). Metastasis and AKT activation. J Cell Physiol. Theodoraki, M. A., Kunijappu, M., Sternberg, D. W. and Caplan, A. J. (2007). Akt shows variable sensitivity to an Hsp90 inhibitor depending on cell context. Exp. Cell. Res. 313, 3851-8.
Xu, W., Yuan, X., Jung, Y. J., Yang, Y., Basso, A., Rosen, N., Chung, E. J., Trepel, J. and Neckers, L. (2003). The heat shock protein 90 inhibitor geldanamycin and the ErbB inhibitor ZD1839 promote rapid PP1 phosphatase-dependent inactivation of AKT in ErbB2 overexpressing breast cancer cells. Cancer Res. 63, 7777-84.
Yang, J., Cron, P., Good, V. M., Thompson, V., Hemmings, B. A. and Barford, D. (2002). Crystal structure of an activated AKT/protein kinase B ternary complex with GSK3-peptide and AMP-PNP. Nat. Struct. Biol. 9, 940-4.
Yuan, T. L. and Cantley, L. C. (2008). PI3K pathway alterations in cancer: variations on a theme. Oncogene. 27, 5497-510.

We claim:

1. Compound 2 of Formula (II): 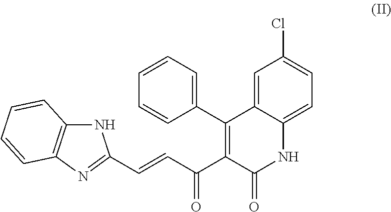
and analogs, derivatives and pharmaceutically acceptable salts thereof.

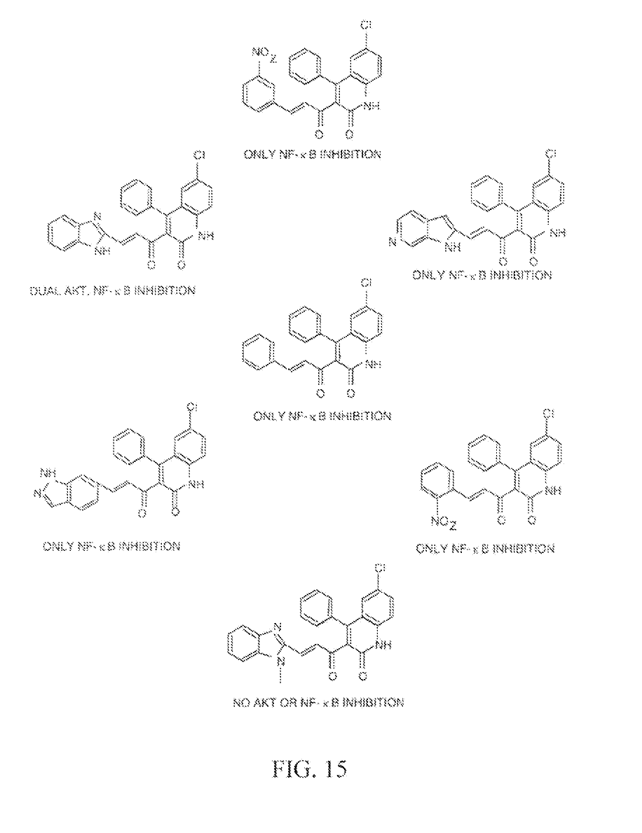

2. The analogs of Compound 2 of Formula (II) according to claim 1, wherein the analogs are selected from compounds 83G10, 98C11, 103F6, 83H2 and 101G9 identified in FIG. 15.

3. A pharmaceutical composition comprising:
Compound 2 of Formula (II) and analogs, derivatives and pharmaceutically acceptable salts thereof; and
a pharmaceutically acceptable carrier.

4. The pharmaceutical composition of claim 3 wherein the composition is effective to inhibit Akt1 kinase activity.

5. The pharmaceutical composition of claim 4 wherein the composition has an IC₅₀ of from less than about 100 μM. to less than about 5 μM.

6. The pharmaceutical composition of claim 3 wherein the composition is effective to inhibit NFkB activity.

7. The pharmaceutical composition of claim 6 wherein the composition has an IC₅₀ of from less than about 100 μM. to less than about 5 μM.

8. A method of treating cancer comprising administering a therapeutically effective amount of Compound 2 of Formula (II) and analogs, derivatives and pharmaceutically acceptable salts and pharmaceutical compositions thereof.

9. The method of claim 8 wherein the cancer comprises melanoma.

10. A method of treating conditions characterized by dysregulation of apoptosis and neurogenerative conditions comprising administering a therapeutically effective amount of Compound 2 of Formula (II) and analogs, derivatives and pharmaceutically acceptable salts and pharmaceutical compositions thereof.

11. A method for decreasing AKT activity in a patient having increased AKT activity or expression, comprising administering a therapeutically effective amount of Compound 2 of Formula (II) and analogs, derivatives and pharmaceutically acceptable salts and pharmaceutical compositions thereof.

12. The method of claim 11 wherein the patient is selected from the group consisting of a human, mouse, rat, dog, cat, sheep, horse, cow, pig, goat, chicken, turkey, duck, and goose.

13. The method of claim 11 wherein said administration is effective to reduce or prevent one or more symptoms of said increased AKT activity.

14. A method for decreasing NFkB activity in a patient having increased NFkB activity or expression, comprising administering a therapeutically effective amount of Compound 2 of Formula (II) and analogs, derivatives and pharmaceutically acceptable salts and pharmaceutical compositions thereof.

15. The method of claim 14 wherein the patient is selected from the group consisting of a human, mouse, rat, dog, cat, sheep, horse, cow, pig, goat, chicken, turkey, duck, and goose.

16. The method of claim 14 wherein said administration is effective to reduce or prevent one or more symptoms of said increased NFkB activity.

17. A kit comprising an amount of Compound 2 of Formula (II) and analogs, derivatives and pharmaceutically acceptable salts and pharmaceutical compositions thereof along with instructions for administering the same to a patient on a label or package insert.

18. A method of preventing cancer in a subject at risk thereof comprising administering to the subject a therapeutically effective amount of Compound 2 of Formula (II), or analogs, derivatives, or pharmaceutically acceptable salts in a pharmaceutically acceptable carrier.

19. The method of claim 18 wherein the cancer is melanoma.
